SEQUENCE LISTING (251 sequences)

SEQ ID NO: 1 - protein, 35 aa, Stichodactyla helianthus
RSCIDTIPKSRCTAFQCKHSMKYRLSFCRKTCGTC

SEQ ID NO: 2 - protein, 35 aa, Artificial Sequence
  ShK-Based Peptide
RSCIDTIPKSRCTAFQSKHSMKYRLSFCRKTSGTC

SEQ ID NO: 3 - protein, 35 aa, Artificial Sequence
  ShK-Based Peptide
RSSIDTIPKSRCTAFQCKHSMKYRLSFCRKTCGTS

SEQ ID NO: 4 - protein, 35 aa, Artificial Sequence
  ShK-Based Peptide
SSCIDTIPKSRCTAFQCKHSMKYRLSFCRKTCGTC

SEQ ID NO: 5 - protein, 35 aa, Artificial Sequence
  ShK-Based Peptide
XSCIDTIPKSRCTAFQCKHSMKYRLSFCRKTCGTC

SEQ ID NO: 6 - protein, 34 aa, Artificial Sequence
  ShK-Based Peptide
SCIDTIPKSRCTAFQCKHSMKYRLSFCRKTCGTC

SEQ ID NO: 7 - protein, 33 aa, Artificial Sequence
  ShK-Based Peptide
CIDTIPKSRCTAFQCKHSMKYRLSFCRKTCGTC

SEQ ID NO: 8 - protein, 35 aa, Artificial Sequence
  ShK-Based Peptide
ASCIDTIPKSRCTAFQCKHSMKYRLSFCRKTCGTC

SEQ ID NO: 9 - protein, 34 aa, Artificial Sequence
  ShK-Based Peptide
QCIDTIPKSRCTAFQCKHSMKYRLSFCRKTCGTC

SEQ ID NO: 10 - protein, 34 aa, Artificial Sequence
  ShK-Based Peptide
ACIDTIPKSRCTAFQCKHSMKYRLSFCRKTCGTC

SEQ ID NO: 11 - protein, 34 aa, Artificial Sequence
  ShK-Based Peptide
TCIDTIPKSRCTAFQCKHSMKYRLSFCRKTCGTC

SEQ ID NO: 12 - protein, 35 aa, Artificial Sequence
  ShK-Based Peptide
RQCIDTIPKSRCTAFQCKHSMKYRLSFCRKTCGTC

SEQ ID NO: 13 - protein, 35 aa, Artificial Sequence
  ShK-Based Peptide
RACIDTIPKSRCTAFQCKHSMKYRLSFCRKTCGTC

SEQ ID NO: 14 - protein, 35 aa, Artificial Sequence
  ShK-Based Peptide
RTCIDTIPKSRCTAFQCKHSMKYRLSFCRKTCGTC

SEQ ID NO: 15 - protein, 35 aa, Artificial Sequence
  ShK-Based Peptide
AQCIDTIPKSRCTAFQCKHSMKYRLSFCRKTCGTC

SEQ ID NO: 16 - protein, 35 aa, Artificial Sequence
  ShK-Based Peptide
AACIDTIPKSRCTAFQCKHSMKYRLSFCRKTCGTC

SEQ ID NO: 17 - protein, 35 aa, Artificial Sequence
  ShK-Based Peptide
ATCIDTIPKSRCTAFQCKHSMKYRLSFCRKTCGTC

SEQ ID NO: 18 - protein, 35 aa, Artificial Sequence
  ShK-Based Peptide
RSCADTIPKSRCTAFQCKHSMKYRLSFCRKTCGTC

SEQ ID NO: 19 - protein, 35 aa, Artificial Sequence
  ShK-Based Peptide
RSCADTIPKSRCTAAQCKHSMKYRLSFCRKTCGTC

SEQ ID NO: 20 - protein, 35 aa, Artificial Sequence
  ShK-Based Peptide
RSCADTIPKSRCTAAQCKHSMKYRASFCRKTCGTC

SEQ ID NO: 21 - protein, 35 aa, Artificial Sequence
  ShK-Based Peptide
RSCIDAIPKSRCTAFQCKHSMKYRLSFCRKTCGTC

SEQ ID NO: 22 - protein, 35 aa, Artificial Sequence; identical to SEQ ID NO: 1
  ShK-Based Peptide

SEQ ID NO: 23 - protein, 35 aa, Artificial Sequence
  ShK-Based Peptide
RSCIDYIPKSRCTAFQCKHSMKYRLSFCRKTCGTC

SEQ ID NO: 24 - protein, 35 aa, Artificial Sequence
  ShK-Based Peptide
RSCIDLIPKSRCTAFQCKHSMKYRLSFCRKTCGTC

SEQ ID NO: 25 - protein, 35 aa, Artificial Sequence
  ShK-Based Peptide
RSCIDTAPKSRCTAFQCKHSMKYRLSFCRKTCGTC

SEQ ID NO: 26 - protein, 35 aa, Artificial Sequence; identical to SEQ ID NO: 18
  ShK-Based Peptide

SEQ ID NO: 27 - protein, 35 aa, Artificial Sequence
  ShK-Based Peptide
RSCIDTIAKSRCTAFQCKHSMKYRLSFCRKTCGTC

SEQ ID NO: 28 - protein, 35 aa, Artificial Sequence
  ShK-Based Peptide
RSCIDTIPASRCTAFQCKHSMKYRLSFCRKTCGTC

SEQ ID NO: 29 - protein, 35 aa, Artificial Sequence
  ShK-Based Peptide
RSCIDTIPESRCTAFQCKHSMKYRLSFCRKTCGTC

SEQ ID NO: 30 - protein, 35 aa, Artificial Sequence
  ShK-Based Peptide
RSCIDTIPQSRCTAFQCKHSMKYRLSFCRKTCGTC

SEQ ID NO: 31 - protein, 35 aa, Artificial Sequence
  ShK-Based Peptide
RSCIDTIPKARCTAFQCKHSMKYRLSFCRKTCGTC

SEQ ID NO: 32 - protein, 35 aa, Artificial Sequence
  ShK-Based Peptide
RSCIDTIPKSACTAFQCKHSMKYRLSFCRKTCGTC

SEQ ID NO: 33 - protein, 35 aa, Artificial Sequence
  ShK-Based Peptide
RSCIDTIPKSECTAFQCKHSMKYRLSFCRKTCGTC

SEQ ID NO: 34 - protein, 35 aa, Artificial Sequence
  ShK-Based Peptide
RSCIDTIPKSQCTAFQCKHSMKYRLSFCRKTCGTC

SEQ ID NO: 35 - protein, 35 aa, Artificial Sequence
  ShK-Based Peptide
RSCIDTIPKSRCAAFQCKHSMKYRLSFCRKTCGTC

SEQ ID NO: 36 - protein, 35 aa, Artificial Sequence
  ShK-Based Peptide
RSCIDTIPKSRCTAAQCKHSMKYRLSFCRKTCGTC

SEQ ID NO: 37 - protein, 35 aa, Artificial Sequence
  ShK-Based Peptide
RSCIDTIPKSRCTAWQCKHSMKYRLSFCRKTCGTC

SEQ ID NO: 38 - protein, 35 aa, Artificial Sequence
  ShK-Based Peptide
RSCIDTIPKSRCTAXQCKHSMKYRLSFCRKTCGTC

SEQ ID NO: 39 - protein, 35 aa, Artificial Sequence
  ShK-Based Peptide
RSCIDTIPKSRCTAAQCKHSMKYRASFCRKTCGTC

SEQ ID NO: 40 - protein, 35 aa, Artificial Sequence
  ShK-Based Peptide
RSCIDTIPKSRCTAFACKHSMKYRLSFCRKTCGTC

SEQ ID NO: 41 - protein, 35 aa, Artificial Sequence
  ShK-Based Peptide
RSCIDTIPKSRCTAFECKHSMKYRLSFCRKTCGTC

SEQ ID NO: 42 - protein, 35 aa, Artificial Sequence
  ShK-Based Peptide
RSCIDTIPKSRCTAFQCAHSMKYRLSFCRKTCGTC

SEQ ID NO: 43 - protein, 35 aa, Artificial Sequence
  ShK-Based Peptide
RSCIDTIPKSRCTAFQCEHSMKYRLSFCRKTCGTC

SEQ ID NO: 44 - protein, 35 aa, Artificial Sequence
  ShK-Based Peptide
RSCIDTIPKSRCTAFQCKASMKYRLSFCRKTCGTC

SEQ ID NO: 45 - protein, 35 aa, Artificial Sequence
  ShK-Based Peptide
RSCIDTIPKSRCTAFQCKKSMKYRLSFCRKTCGTC

SEQ ID NO: 46 - protein, 35 aa, Artificial Sequence
  ShK-Based Peptide
RSCIDTIPKSRCTAFQCKHAMKYRLSFCRKTCGTC

SEQ ID NO: 47 - protein, 35 aa, Artificial Sequence
  ShK-Based Peptide
RSCIDTIPKSRCTAFQCKHSAKYRLSFCRKTCGTC

SEQ ID NO: 48 - protein, 35 aa, Artificial Sequence
  ShK-Based Peptide
RSCIDTIPKSRCTAFQCKHSXKYRLSFCRKTCGTC

SEQ ID NO: 49 - protein, 35 aa, Artificial Sequence; identical to SEQ ID NO: 48
  ShK-Based Peptide

SEQ ID NO: 50 - protein, 35 aa, Artificial Sequence
  ShK-Based Peptide
RSCIDTIPKSRCTAFQCKHSMAYRLSFCRKTCGTC

SEQ ID NO: 51 - protein, 35 aa, Artificial Sequence
  ShK-Based Peptide
RSCIDTIPKSRCTAFQCKHSMEYRLSFCRKTCGTC

SEQ ID NO: 52 - protein, 35 aa, Artificial Sequence
  ShK-Based Peptide
RSCIDTIPKSRCTAFQCKHSMRYRLSFCRKTCGTC

SEQ ID NO: 53 - protein, 35 aa, Artificial Sequence
  ShK-Based Peptide
RSCIDTIPKSRCTAFQCKHSMXYRLSFCRKTCGTC

SEQ ID NO: 54 - protein, 35 aa, Artificial Sequence; identical to SEQ ID NO: 53
  ShK-Based Peptide

SEQ ID NO: 55 - protein, 35 aa, Artificial Sequence; identical to SEQ ID NO: 53
  ShK-Based Peptide

SEQ ID NO: 56 - protein, 35 aa, Artificial Sequence; identical to SEQ ID NO: 53
  ShK-Based Peptide

SEQ ID NO: 57 - protein, 35 aa, Artificial Sequence; identical to SEQ ID NO: 53
  ShK-Based Peptide

SEQ ID NO: 58 - protein, 35 aa, Artificial Sequence
  ShK-Based Peptide
RSCIDTIPKSRCTAFQCKHSMKARLSFCRKTCGTC

SEQ ID NO: 59 - protein, 35 aa, Artificial Sequence
  ShK-Based Peptide
RSCIDTIPKSRCTAFQCKHSMKSRLSFCRKTCGTC

SEQ ID NO: 60 - protein, 35 aa, Artificial Sequence
  ShK-Based Peptide
RSCIDTIPKSRCTAFQCKHSMKFRLSFCRKTCGTC

SEQ ID NO: 61 - protein, 35 aa, Artificial Sequence
  ShK-Based Peptide
RSCIDTIPKSRCTAFQCKHSMKXRLSFCRKTCGTC

SEQ ID NO: 62 - protein, 35 aa, Artificial Sequence; identical to SEQ ID NO: 61
  ShK-Based Peptide

SEQ ID NO: 63 - protein, 36 aa, Artificial Sequence
  ShK-Based Peptide
RSCIDTIPKSRCTAFQCKHSMKXRLSFCRKTCGTCC

SEQ ID NO: 64 - protein, 35 aa, Artificial Sequence; identical to SEQ ID NO: 61
  ShK-Based Peptide

SEQ ID NO: 65 - protein, 35 aa, Artificial Sequence
  ShK-Based Peptide
RSCIDTIPKSRCTAFQCKHSMKYALSFCRKTCGTC

SEQ ID NO: 66 - protein, 35 aa, Artificial Sequence
  ShK-Based Peptide
RSCIDTIPKSRCTAFQCKHSMKYELSFCRKTCGTC

SEQ ID NO: 67 - protein, 35 aa, Artificial Sequence
  ShK-Based Peptide
RSCIDTIPKSRCTAFQCKHSMKYRASFCRKTCGTC

SEQ ID NO: 68 - protein, 35 aa, Artificial Sequence
  ShK-Based Peptide
RSCIDTIPKSRCTAFQCKHSMKYRLAFCRKTCGTC

SEQ ID NO: 69 - protein, 35 aa, Artificial Sequence
  ShK-Based Peptide
RSCIDTIPKSRCTAFQCKHSMKYRLSACRKTCGTC

SEQ ID NO: 70 - protein, 35 aa, Artificial Sequence
  ShK-Based Peptide
RSCIDTIPKSRCTAFQCKHSMKYRLSXCRKTCGTC

SEQ ID NO: 71 - protein, 35 aa, Artificial Sequence
  ShK-Based Peptide
RSCIDTIPKSRCTAFQCKHSMKYRLSFCAKTCGTC

SEQ ID NO: 72 - protein, 35 aa, Artificial Sequence
  ShK-Based Peptide
RSCIDTIPKSRCTAFQCKHSMKYRLSFCRATCGTC

SEQ ID NO: 73 - protein, 35 aa, Artificial Sequence
  ShK-Based Peptide
RSCIDTIPKSRCTAFQCKHSMKYRLSFCRKACGTC

SEQ ID NO: 74 - protein, 35 aa, Artificial Sequence
  ShK-Based Peptide
RSCIDTIPKSRCTAFQCKHSMKYRLSFCRKTCGAC

SEQ ID NO: 75 - protein, 34 aa, Artificial Sequence
  ShK-Based Peptide
SCADTIPKSRCTAFQCKHSMKYRLSFCRKTCGTC

SEQ ID NO: 76 - protein, 34 aa, Artificial Sequence
  ShK-Based Peptide
SCADTIPKSRCTAAQCKHSMKYRLSFCRKTCGTC

SEQ ID NO: 77 - protein, 34 aa, Artificial Sequence
  ShK-Based Peptide
SCADTIPKSRCTAAQCKHSMKYRASFCRKTCGTC

SEQ ID NO: 78 - protein, 34 aa, Artificial Sequence
  ShK-Based Peptide
SCIDAIPKSRCTAFQCKHSMKYRLSFCRKTCGTC

SEQ ID NO: 79 - protein, 34 aa, Artificial Sequence
  ShK-Based Peptide
SCIDTAPKSRCTAFQCKHSMKYRLSFCRKTCGTC

SEQ ID NO: 80 - protein, 34 aa, Artificial Sequence
  ShK-Based Peptide
SCIDTIAKSRCTAFQCKHSMKYRLSFCRKTCGTC

SEQ ID NO: 81 - protein, 34 aa, Artificial Sequence
  ShK-Based Peptide
SCIDTIPASRCTAFQCKHSMKYRLSFCRKTCGTC

SEQ ID NO: 82 - protein, 34 aa, Artificial Sequence
  ShK-Based Peptide
SCIDTIPESRCTAFQCKHSMKYRLSFCRKTCGTC

SEQ ID NO: 83 - protein, 34 aa, Artificial Sequence
  ShK-Based Peptide
SCIDTIPQSRCTAFQCKHSMKYRLSFCRKTCGTC

SEQ ID NO: 84 - protein, 34 aa, Artificial Sequence
  ShK-Based Peptide
SCIDTIPKARCTAFQCKHSMKYRLSFCRKTCGTC

SEQ ID NO: 85 - protein, 34 aa, Artificial Sequence
  ShK-Based Peptide
SCIDTIPKSACTAFQCKHSMKYRLSFCRKTCGTC

SEQ ID NO: 86 - protein, 34 aa, Artificial Sequence
  ShK-Based Peptide
SCIDTIPKSECTAFQCKHSMKYRLSFCRKTCGTC

SEQ ID NO: 87 - protein, 34 aa, Artificial Sequence
  ShK-Based Peptide
SCIDTIPKSQCTAFQCKHSMKYRLSFCRKTCGTC

SEQ ID NO: 88 - protein, 34 aa, Artificial Sequence
  ShK-Based Peptide
SCIDTIPKSRCAAFQCKHSMKYRLSFCRKTCGTC

SEQ ID NO: 89 - protein, 34 aa, Artificial Sequence
  ShK-Based Peptide
SCIDTIPKSRCTAAQCKHSMKYRLSFCRKTCGTC

SEQ ID NO: 90 - protein, 34 aa, Artificial Sequence
  ShK-Based Peptide
SCIDTIPKSRCTAWQCKHSMKYRLSFCRKTCGTC

SEQ ID NO: 91 - protein, 34 aa, Artificial Sequence
  ShK-Based Peptide
SCIDTIPKSRCTAXQCKHSMKYRLSFCRKTCGTC

SEQ ID NO: 92 - protein, 34 aa, Artificial Sequence
  ShK-Based Peptide
SCIDTIPKSRCTAAQCKHSMKYRASFCRKTCGTC

SEQ ID NO: 93 - protein, 34 aa, Artificial Sequence
  ShK-Based Peptide
SCIDTIPKSRCTAFACKHSMKYRLSFCRKTCGTC

SEQ ID NO: 94 - protein, 34 aa, Artificial Sequence
  ShK-Based Peptide
SCIDTIPKSRCTAFECKHSMKYRLSFCRKTCGTC

SEQ ID NO: 95 - protein, 34 aa, Artificial Sequence
  ShK-Based Peptide
SCIDTIPKSRCTAFQCAHSMKYRLSFCRKTCGTC

SEQ ID NO: 96 - protein, 34 aa, Artificial Sequence
  ShK-Based Peptide
SCIDTIPKSRCTAFQCEHSMKYRLSFCRKTCGTC

SEQ ID NO: 97 - protein, 34 aa, Artificial Sequence
  ShK-Based Peptide
SCIDTIPKSRCTAFQCKASMKYRLSFCRKTCGTC

SEQ ID NO: 98 - protein, 34 aa, Artificial Sequence
  ShK-Based Peptide
SCIDTIPKSRCTAFQCKKSMKYRLSFCRKTCGTC

SEQ ID NO: 99 - protein, 34 aa, Artificial Sequence
  ShK-Based Peptide
SCIDTIPKSRCTAFQCKHAMKYRLSFCRKTCGTC

SEQ ID NO: 100 - protein, 34 aa, Artificial Sequence
  ShK-Based Peptide
SCIDTIPKSRCTAFQCKHSAKYRLSFCRKTCGTC

SEQ ID NO: 101 - protein, 34 aa, Artificial Sequence
  ShK-Based Peptide
SCIDTIPKSRCTAFQCKHSXKYRLSFCRKTCGTC

SEQ ID NO: 102 - protein, 34 aa, Artificial Sequence; identical to SEQ ID NO: 101
  ShK-Based Peptide

SEQ ID NO: 103 - protein, 34 aa, Artificial Sequence
  ShK-Based Peptide
SCIDTIPKSRCTAFQCKHSMAYRLSFCRKTCGTC

SEQ ID NO: 104 - protein, 34 aa, Artificial Sequence
  ShK-Based Peptide
SCIDTIPKSRCTAFQCKHSMEYRLSFCRKTCGTC

SEQ ID NO: 105 - protein, 34 aa, Artificial Sequence
  ShK-Based Peptide
SCIDTIPKSRCTAFQCKHSMRYRLSFCRKTCGTC

SEQ ID NO: 106 - protein, 34 aa, Artificial Sequence
  ShK-Based Peptide
SCIDTIPKSRCTAFQCKHSMXYRLSFCRKTCGTC

SEQ ID NO: 107 - protein, 34 aa, Artificial Sequence; identical to SEQ ID NO: 106
  ShK-Based Peptide

SEQ ID NO: 108 - protein, 34 aa, Artificial Sequence; identical to SEQ ID NO: 106
  ShK-Based Peptide

SEQ ID NO: 109 - protein, 34 aa, Artificial Sequence; identical to SEQ ID NO: 106
  ShK-Based Peptide

SEQ ID NO: 110 - protein, 34 aa, Artificial Sequence; identical to SEQ ID NO: 106
  ShK-Based Peptide

SEQ ID NO: 111 - protein, 34 aa, Artificial Sequence
  ShK-Based Peptide
SCIDTIPKSRCTAFQCKHSMKARLSFCRKTCGTC

SEQ ID NO: 112 - protein, 34 aa, Artificial Sequence
  ShK-Based Peptide
SCIDTIPKSRCTAFQCKHSMKSRLSFCRKTCGTC

SEQ ID NO: 113 - protein, 34 aa, Artificial Sequence
  ShK-Based Peptide
SCIDTIPKSRCTAFQCKHSMKFRLSFCRKTCGTC

SEQ ID NO: 114 - protein, 34 aa, Artificial Sequence
  ShK-Based Peptide
SCIDTIPKSRCTAFQCKHSMKXRLSFCRKTCGTC

SEQ ID NO: 115 - protein, 34 aa, Artificial Sequence; identical to SEQ ID NO: 114
  ShK-Based Peptide

SEQ ID NO: 116 - protein, 34 aa, Artificial Sequence; identical to SEQ ID NO: 114
  ShK-Based Peptide

SEQ ID NO: 117 - protein, 33 aa, Artificial Sequence
  ShK-Based Peptide
SCIDTIPKSRCTAFQCKHSMKXRLSFCRKTCGT

SEQ ID NO: 118 - protein, 34 aa, Artificial Sequence
  ShK-Based Peptide
SCIDTIPKSRCTAFQCKHSMKYALSFCRKTCGTC

SEQ ID NO: 119 - protein, 34 aa, Artificial Sequence
  ShK-Based Peptide
SCIDTIPKSRCTAFQCKHSMKYELSFCRKTCGTC

SEQ ID NO: 120 - protein, 34 aa, Artificial Sequence
  ShK-Based Peptide
SCIDTIPKSRCTAFQCKHSMKYRASFCRKTCGTC

SEQ ID NO: 121 - protein, 34 aa, Artificial Sequence
  ShK-Based Peptide
SCIDTIPKSRCTAFQCKHSMKYRLAFCRKTCGTC

SEQ ID NO: 122 - protein, 34 aa, Artificial Sequence
  ShK-Based Peptide
SCIDTIPKSRCTAFQCKHSMKYRLSACRKTCGTC

SEQ ID NO: 123 - protein, 34 aa, Artificial Sequence
  ShK-Based Peptide
SCIDTIPKSRCTAFQCKHSMKYRLSXCRKTCGTC

SEQ ID NO: 124 - protein, 34 aa, Artificial Sequence
  ShK-Based Peptide
SCIDTIPKSRCTAFQCKHSMKYRLSFCAKTCGTC

SEQ ID NO: 125 - protein, 34 aa, Artificial Sequence
  ShK-Based Peptide
SCIDTIPKSRCTAFQCKHSMKYRLSFCRATCGTC

SEQ ID NO: 126 - protein, 34 aa, Artificial Sequence
  ShK-Based Peptide
SCIDTIPKSRCTAFQCKHSMKYRLSFCRKACGTC

SEQ ID NO: 127 - protein, 34 aa, Artificial Sequence
  ShK-Based Peptide
SCIDTIPKSRCTAFQCKHSMKYRLSFCRKTCGAC

SEQ ID NO: 128 - protein, 35 aa, Artificial Sequence
  ShK-Based Peptide
YSCIDTIPKSRCTAFQCKHSMKYRLSFCRKTCGTC

SEQ ID NO: 129 - protein, 35 aa, Artificial Sequence
  ShK-Based Peptide
KSCIDTIPKSRCTAFQCKHSMKYRLSFCRKTCGTC

SEQ ID NO: 130 - protein, 35 aa, Artificial Sequence
  ShK-Based Peptide
HSCIDTIPKSRCTAFQCKHSMKYRLSFCRKTCGTC

SEQ ID NO: 131 - protein, 35 aa, Artificial Sequence
  ShK-Based Peptide
QSCIDTIPKSRCTAFQCKHSMKYRLSFCRKTCGTC

SEQ ID NO: 132 - protein, 37 aa, Artificial Sequence
  ShK-Based Peptide
PPRSCIDTIPKSRCTAFQCKHSMKYRLSFCRKTCGTC

SEQ ID NO: 133 - protein, 36 aa, Artificial Sequence
  ShK-Based Peptide
MRSCIDTIPKSRCTAFQCKHSMKYRLSFCRKTCGTC

SEQ ID NO: 134 - protein, 36 aa, Artificial Sequence
  ShK-Based Peptide
GRSCIDTIPKSRCTAFQCKHSMKYRLSFCRKTCGTC

SEQ ID NO: 135 - protein, 35 aa, Artificial Sequence
  ShK-Based Peptide
YSCIDTIPKSRCTAFQCKHSMAYRLSFCRKTCGTC

SEQ ID NO: 136 - protein, 35 aa, Artificial Sequence
  ShK-Based Peptide
KSCIDTIPKSRCTAFQCKHSMAYRLSFCRKTCGTC

SEQ ID NO: 137 - protein, 35 aa, Artificial Sequence
  ShK-Based Peptide
HSCIDTIPKSRCTAFQCKHSMAYRLSFCRKTCGTC

SEQ ID NO: 138 - protein, 35 aa, Artificial Sequence
  ShK-Based Peptide
QSCIDTIPKSRCTAFQCKHSMAYRLSFCRKTCGTC

SEQ ID NO: 139 - protein, 37 aa, Artificial Sequence
  ShK-Based Peptide
PPRSCIDTIPKSRCTAFQCKHSMAYRLSFCRKTCGTC

SEQ ID NO: 140 - protein, 36 aa, Artificial Sequence
  ShK-Based Peptide
MRSCIDTIPKSRCTAFQCKHSMAYRLSFCRKTCGTC

SEQ ID NO: 141 - protein, 36 aa, Artificial Sequence
  ShK-Based Peptide
GRSCIDTIPKSRCTAFQCKHSMAYRLSFCRKTCGTC

SEQ ID NO: 142 - protein, 35 aa, Artificial Sequence
  ShK-Based Peptide
RSCIDTIPASRCTAFQCKHSMAYRLSFCRKTCGTC

SEQ ID NO: 143 - protein, 34 aa, Artificial Sequence
  ShK-Based Peptide
SCIDTIPASRCTAFQCKHSMAYRLSFCRKTCGTC

SEQ ID NO: 144 - protein, 35 aa, Artificial Sequence
  ShK-Based Peptide
RSCIDTIPVSRCTAFQCKHSMKYRLSFCRKTCGTC

SEQ ID NO: 145 - protein, 35 aa, Artificial Sequence
  ShK-Based Peptide
RSCIDTIPVSRCTAFQCKHSMAYRLSFCRKTCGTC

SEQ ID NO: 146 - protein, 34 aa, Artificial Sequence
  ShK-Based Peptide
SCIDTIPVSRCTAFQCKHSMKYRLSFCRKTCGTC

SEQ ID NO: 147 - protein, 34 aa, Artificial Sequence
  ShK-Based Peptide
SCIDTIPVSRCTAFQCKHSMAYRLSFCRKTCGTC

SEQ ID NO: 148 - protein, 35 aa, Artificial Sequence
  ShK-Based Peptide
RSCIDTIPESRCTAFQCKHSMAYRLSFCRKTCGTC

SEQ ID NO: 149 - protein, 34 aa, Artificial Sequence
  ShK-Based Peptide
SCIDTIPESRCTAFQCKHSMAYRLSFCRKTCGTC

SEQ ID NO: 150 - protein, 35 aa, Artificial Sequence
  ShK-Based Peptide
RSCIDTIPKSACTAFQCKHSMAYRLSFCRKTCGTC

SEQ ID NO: 151 - protein, 34 aa, Artificial Sequence
  ShK-Based Peptide
SCIDTIPKSACTAFQCKHSMAYRLSFCRKTCGTC

SEQ ID NO: 152 - protein, 35 aa, Artificial Sequence
  ShK-Based Peptide
RSCIDTIPKSECTAFQCKHSMAYRLSFCRKTCGTC

SEQ ID NO: 153 - protein, 34 aa, Artificial Sequence
  ShK-Based Peptide
SCIDTIPKSECTAFQCKHSMAYRLSFCRKTCGTC

SEQ ID NO: 154 - protein, 35 aa, Artificial Sequence
  ShK-Based Peptide
RSCIDTIPKSRCTDFQCKHSMKYRLSFCRKTCGTC

SEQ ID NO: 155 - protein, 35 aa, Artificial Sequence
  ShK-Based Peptide
RSCIDTIPKSRCTDFQCKHSMAYRLSFCRKTCGTC

SEQ ID NO: 156 - protein, 34 aa, Artificial Sequence
  ShK-Based Peptide
SCIDTIPKSRCTDFQCKHSMKYRLSFCRKTCGTC

SEQ ID NO: 157 - protein, 34 aa, Artificial Sequence
  ShK-Based Peptide
SCIDTIPKSRCTDFQCKHSMAYRLSFCRKTCGTC

SEQ ID NO: 158 - protein, 35 aa, Artificial Sequence
  ShK-Based Peptide
RSCIDTIPKSRCTAAQCKHSMAYRLSFCRKTCGTC

SEQ ID NO: 159 - protein, 34 aa, Artificial Sequence
  ShK-Based Peptide
SCIDTIPKSRCTAAQCKHSMAYRLSFCRKTCGTC

SEQ ID NO: 160 - protein, 35 aa, Artificial Sequence
  ShK-Based Peptide
RSCIDTIPKSRCTAIQCKHSMKYRLSFCRKTCGTC

SEQ ID NO: 161 - protein, 35 aa, Artificial Sequence
  ShK-Based Peptide
RSCIDTIPKSRCTAIQCKHSMAYRLSFCRKTCGTC

SEQ ID NO: 162 - protein, 34 aa, Artificial Sequence
  ShK-Based Peptide
SCIDTIPKSRCTAIQCKHSMKYRLSFCRKTCGTC

SEQ ID NO: 163 - protein, 34 aa, Artificial Sequence
  ShK-Based Peptide
SCIDTIPKSRCTAIQCKHSMAYRLSFCRKTCGTC

SEQ ID NO: 164 - protein, 35 aa, Artificial Sequence
  ShK-Based Peptide
RSCIDTIPKSRCTAVQCKHSMKYRLSFCRKTCGTC

SEQ ID NO: 165 - protein, 35 aa, Artificial Sequence
  ShK-Based Peptide
RSCIDTIPKSRCTAVQCKHSMAYRLSFCRKTCGTC

SEQ ID NO: 166 - protein, 34 aa, Artificial Sequence
  ShK-Based Peptide
SCIDTIPKSRCTAVQCKHSMKYRLSFCRKTCGTC

SEQ ID NO: 167 - protein, 34 aa, Artificial Sequence
  ShK-Based Peptide
SCIDTIPKSRCTAVQCKHSMAYRLSFCRKTCGTC

SEQ ID NO: 168 - protein, 35 aa, Artificial Sequence
  ShK-Based Peptide
RSCIDTIPKSRCTAFRCKHSMKYRLSFCRKTCGTC

SEQ ID NO: 169 - protein, 35 aa, Artificial Sequence
  ShK-Based Peptide
RSCIDTIPKSRCTAFRCKHSMAYRLSFCRKTCGTC

SEQ ID NO: 170 - protein, 34 aa, Artificial Sequence
  ShK-Based Peptide
SCIDTIPKSRCTAFRCKHSMKYRLSFCRKTCGTC

SEQ ID NO: 171 - protein, 34 aa, Artificial Sequence
  ShK-Based Peptide
SCIDTIPKSRCTAFRCKHSMAYRLSFCRKTCGTC

SEQ ID NO: 172 - protein, 35 aa, Artificial Sequence
  ShK-Based Peptide
RSCIDTIPKSRCTAFKCKHSMKYRLSFCRKTCGTC

SEQ ID NO: 173 - protein, 35 aa, Artificial Sequence
  ShK-Based Peptide
RSCIDTIPKSRCTAFKCKHSMAYRLSFCRKTCGTC

SEQ ID NO: 174 - protein, 34 aa, Artificial Sequence
  ShK-Based Peptide
SCIDTIPKSRCTAFKCKHSMKYRLSFCRKTCGTC

SEQ ID NO: 175 - protein, 34 aa, Artificial Sequence
  ShK-Based Peptide
SCIDTIPKSRCTAFKCKHSMAYRLSFCRKTCGTC

SEQ ID NO: 176 - protein, 35 aa, Artificial Sequence
  ShK-Based Peptide
RSCIDTIPASECTAFQCKHSMKYRLSFCRKTCGTC

SEQ ID NO: 177 - protein, 35 aa, Artificial Sequence
  ShK-Based Peptide
RSCIDTIPASECTAFQCKHSMAYRLSFCRKTCGTC

SEQ ID NO: 178 - protein, 34 aa, Artificial Sequence
  ShK-Based Peptide
SCIDTIPASECTAFQCKHSMKYRLSFCRKTCGTC

SEQ ID NO: 179 - protein, 34 aa, Artificial Sequence
  ShK-Based Peptide
SCIDTIPASECTAFQCKHSMAYRLSFCRKTCGTC

SEQ ID NO: 180 - protein, 35 aa, Artificial Sequence
  ShK-Based Peptide
RSCIDTIPVSECTAFQCKHSMKYRLSFCRKTCGTC

SEQ ID NO: 181 - protein, 35 aa, Artificial Sequence
  ShK-Based Peptide
RSCIDTIPVSECTAFQCKHSMAYRLSFCRKTCGTC

SEQ ID NO: 182 - protein, 34 aa, Artificial Sequence
  ShK-Based Peptide
SCIDTIPVSECTAFQCKHSMKYRLSFCRKTCGTC

SEQ ID NO: 183 - protein, 34 aa, Artificial Sequence
  ShK-Based Peptide
SCIDTIPVSECTAFQCKHSMAYRLSFCRKTCGTC

SEQ ID NO: 184 - protein, 35 aa, Artificial Sequence
  ShK-Based Peptide
RSCIDTIPVSACTAFQCKHSMKYRLSFCRKTCGTC

SEQ ID NO: 185 - protein, 35 aa, Artificial Sequence
  ShK-Based Peptide
RSCIDTIPVSACTAFQCKHSMAYRLSFCRKTCGTC

SEQ ID NO: 186 - protein, 34 aa, Artificial Sequence
  ShK-Based Peptide
SCIDTIPVSACTAFQCKHSMKYRLSFCRKTCGTC

SEQ ID NO: 187 - protein, 34 aa, Artificial Sequence
  ShK-Based Peptide
SCIDTIPVSACTAFQCKHSMAYRLSFCRKTCGTC

SEQ ID NO: 188 - protein, 35 aa, Artificial Sequence
  ShK-Based Peptide
RSCIDTIPASACTAFQCKHSMKYRLSFCRKTCGTC

SEQ ID NO: 189 - protein, 35 aa, Artificial Sequence
  ShK-Based Peptide
RSCIDTIPASACTAFQCKHSMAYRLSFCRKTCGTC

SEQ ID NO: 190 - protein, 34 aa, Artificial Sequence
  ShK-Based Peptide
SCIDTIPASACTAFQCKHSMKYRLSFCRKTCGTC

SEQ ID NO: 191 - protein, 34 aa, Artificial Sequence
  ShK-Based Peptide
SCIDTIPASACTAFQCKHSMAYRLSFCRKTCGTC

SEQ ID NO: 192 - protein, 35 aa, Artificial Sequence
  ShK-Based Peptide
RSCIDTIPKSECTDIRCKHSMKYRLSFCRKTCGTC

SEQ ID NO: 193 - protein, 35 aa, Artificial Sequence
  ShK-Based Peptide
RSCIDTIPKSECTDIRCKHSMAYRLSFCRKTCGTC

SEQ ID NO: 194 - protein, 34 aa, Artificial Sequence
  ShK-Based Peptide
SCIDTIPKSECTDIRCKHSMKYRLSFCRKTCGTC

SEQ ID NO: 195 - protein, 34 aa, Artificial Sequence
  ShK-Based Peptide
SCIDTIPKSECTDIRCKHSMAYRLSFCRKTCGTC

SEQ ID NO: 196 - protein, 35 aa, Artificial Sequence
  ShK-Based Peptide
RSCIDTIPVSECTDIRCKHSMKYRLSFCRKTCGTC

SEQ ID NO: 197 - protein, 35 aa, Artificial Sequence
  ShK-Based Peptide
RSCIDTIPVSECTDIRCKHSMAYRLSFCRKTCGTC

SEQ ID NO: 198 - protein, 34 aa, Artificial Sequence
  ShK-Based Peptide
SCIDTIPVSECTDIRCKHSMKYRLSFCRKTCGTC

SEQ ID NO: 199 - protein, 34 aa, Artificial Sequence
  ShK-Based Peptide
SCIDTIPVSECTDIRCKHSMAYRLSFCRKTCGTC

SEQ ID NO: 200 - protein, 35 aa, Artificial Sequence
  ShK-Based Peptide
RSCIDTIPVSECTDIQCKHSMKYRLSFCRKTCGTC

SEQ ID NO: 201 - protein, 35 aa, Artificial Sequence
  ShK-Based Peptide
RSCIDTIPVSECTDIQCKHSMAYRLSFCRKTCGTC

SEQ ID NO: 202 - protein, 34 aa, Artificial Sequence
  ShK-Based Peptide
SCIDTIPVSECTDIQCKHSMKYRLSFCRKTCGTC

SEQ ID NO: 203 - protein, 34 aa, Artificial Sequence
  ShK-Based Peptide
SCIDTIPVSECTDIQCKHSMAYRLSFCRKTCGTC

SEQ ID NO: 204 - protein, 35 aa, Artificial Sequence
  ShK-Based Peptide
RTCKDLIPVSECTDIRCKHSMKYRLSFCRKTCGTC

SEQ ID NO: 205 - protein, 35 aa, Artificial Sequence
  ShK-Based Peptide
RTCKDLIPVSECTDIRCKHSMAYRLSFCRKTCGTC

SEQ ID NO: 206 - protein, 34 aa, Artificial Sequence
  ShK-Based Peptide
TCKDLIPVSECTDIRCKHSMKYRLSFCRKTCGTC

SEQ ID NO: 207 - protein, 34 aa, Artificial Sequence
  ShK-Based Peptide
TCKDLIPVSECTDIRCKHSMAYRLSFCRKTCGTC

SEQ ID NO: 208 - protein, 37 aa, Artificial Sequence
  ShK-Based Peptide
XXRSCIDTIPKSRCTAFQCKHSMKYRLSFCRKTCGTC

SEQ ID NO: 209 - protein, 37 aa, Artificial Sequence; identical to SEQ ID NO: 208
  ShK-Based Peptide

SEQ ID NO: 210 - protein, 37 aa, Artificial Sequence; identical to SEQ ID NO: 208
  ShK-Based Peptide

SEQ ID NO: 211 - protein, 35 aa, Artificial Sequence
  ShK-Based Peptide
QSCADTIPKSRCTAAQCKHSMKYRLSFCRKTCGTC

SEQ ID NO: 212 - protein, 35 aa, Artificial Sequence
  ShK-Based Peptide
QSCADTIPKSRCTAAQCKHSMAYRLSFCRKTCGTC

SEQ ID NO: 213 - protein, 35 aa, Artificial Sequence
  ShK-Based Peptide
QSCADTIPKSRCTAAQCKHSMXYRLSFCRKTCGTC

SEQ ID NO: 214 - protein, 35 aa, Artificial Sequence
  ShK-Based Peptide
QSCADTIPKSRCTAAQCKHSMKYRASFCRKTCGTC

SEQ ID NO: 215 - protein, 35 aa, Artificial Sequence
  ShK-Based Peptide
QSCADTIPKSRCTAAQCKHSMAYRASFCRKTCGTC

SEQ ID NO: 216 - protein, 35 aa, Artificial Sequence
  ShK-Based Peptide
QSCADTIPKSRCTAAQCKHSMXYRASFCRKTCGTC

SEQ ID NO: 217 - protein, 37 aa, Artificial Sequence; identical to SEQ ID NO: 208
  ShK-Based Peptide

SEQ ID NO: 218 - protein, 37 aa, Artificial Sequence
  ShK-Based Peptide
XXRSCIDTIPKSRCTAFQCKHSXKYRLSFCRKTCGTC

SEQ ID NO: 219 - protein, 37 aa, Artificial Sequence; identical to SEQ ID NO: 208
  ShK-Based Peptide

SEQ ID NO: 220 - protein, 37 aa, Artificial Sequence; identical to SEQ ID NO: 218
  ShK-Based Peptide

SEQ ID NO: 221 - protein, 39 aa, Artificial Sequence
  ShK-Based Peptide
XXXXRSCIDTIPKSRCTAFQCKHSMKYRLSFCRKTCGTC

SEQ ID NO: 222 - protein, 37 aa, Artificial Sequence
  ShK-Based Peptide
XXRSCIDTIPKSRCTAFKCKHSXKYRLSFCRKTCGTC

SEQ ID NO: 223 - protein, 37 aa, Artificial Sequence; identical to SEQ ID NO: 208
  ShK-Based Peptide

SEQ ID NO: 224 - protein, 35 aa, Artificial Sequence
  ShK-Based Peptide
RSCIDTIPKSRCTAFQCKHSXXYRLSFCRKTCGTC

SEQ ID NO: 225 - protein, 35 aa, Pandinus imperator
LVKCRGTSDCGRPCQQQTGCPNSKCINRMCKCYGC

SEQ ID NO: 226 - protein, 35 aa, Pandinus imperator
TISCTNPKQCYPHCKKETGYPNAKCMNRKCKCFGR

SEQ ID NO: 227 - protein, 35 aa, Pandinus imperator
TISCTNEKQCYPHCKKETGYPNAKCMNRKCKCFGR

SEQ ID NO: 228 - protein, 38 aa, Pandinus imperator
IEAIRCGGSRDCYRPCQKRTGCPNAKCINKTCKCYGCS

SEQ ID NO: 229 - protein, 34 aa, Heterometrus spinifer
ASCRTPKDCADPCRKETGCPYGKCMNRKCKCNRC

SEQ ID NO: 230 - protein, 38 aa, Leiurus quinquestriatus hebraeus
GVPINVSCTGSPQCIKPCKDAGMRFGKCMNRKCHCTPK

SEQ ID NO: 231 - protein, 38 aa, Leiurus quinquestriatus hebraeus
GVPINVKCTGSPQCLKPCKDAGMRFGKCINGKCHCTPK

SEQ ID NO: 232 - protein, 38 aa, Orthochirus scrobiculosus
GVIINVKCKISRQCLEPCKKAGMRFGKCMNGKCHCTPK

SEQ ID NO: 233 - protein, 35 aa, Anuroctonus phaiodactylus
ZKECTGPQHCTNFCRKNKCTHGKCMNRKCKCFNCK

SEQ ID NO: 234 - protein, 39 aa, Centruroides noxius
TIINVKCTSPKQCSKPCKELYGSSAGAKCMNGKCKCYNN

SEQ ID NO: 235 - protein, 39 aa, Centruroides limbatus
TVIDVKCTSPKQCLPPCKAQFGLRAGAKCMNGKCKCYPH

SEQ ID NO: 236 - protein, 37 aa, Leiurus quinquestriatus hebraeus
QFTNVSCTTSKECWSVCQRLHNTSRGKCMNKKCRCYS

SEQ ID NO: 237 - protein, 37 aa, Tityus serrulatus
VFINAKCRGSPECLPKCKEAIGKAAGKCMNGKCKCYP

SEQ ID NO: 238 - protein, 37 aa, Bunodosoma granuliferum
VCRDWFKETACRHAKSLGNCRTSQKYRANCAKTCELC

SEQ ID NO: 239 - protein, 38 aa, Mesobuthus martensii
VGINVKCKHSGQCLKPCKDAGMRFGKCINGKCDCTPKG

SEQ ID NO: 240 - protein, 37 aa, Mesobuthus martensii
QFTDVKCTGSKQCWPVCKQMFGKPNGKCMNGKCRCYS

SEQ ID NO: 241 - protein, 37 aa, Tityus cambridgei
VFINVKCRGSKECLPACKAAVGKAAGKCMNGKCKCYP

SEQ ID NO: 242 - protein, 35 aa, Tityus cambridgei
TGPQTTCQAAMCEAGCKGLGKSMESCQGDTCKCKA

SEQ ID NO: 243 - protein, 36 aa, Vaejovis mexicanus smithi
  MOD_RES (36)..(36): AMIDATION
AAAISCVGSPECPPKCRAQGCKNGKCMNRKCKCYYC

SEQ ID NO: 244 - protein, 35 aa, Heteractis magnifica
RTCKDLIPVSECTDIRCRTSMKYRLNLCRKTCGSC

SEQ ID NO: 245 - protein, 36 aa, Actinia equina
GCKDNFSANTCKHVKANNNCGSQKYATNCAKTCGKC

SEQ ID NO: 246 - protein, 36 aa, Anemonia sulcata
ACKDNFAAATCKHVKENKNCGSQKYATNCAKTCGKC

SEQ ID NO: 247 - protein, 39 aa, Centruroides margaritatus
TIINVKCTSPKQCLPPCKAQFGQSAGAKCMNGKCKCYPH

SEQ ID NO: 248 - protein, 38 aa, Androctonus mauritanicus mauritanicus
GVEINVKCSGSPQCLKPCKDAGMRFGKCMNRKCHCTPK

SEQ ID NO: 249 - protein, 37 aa, Androctonus australis
VRIPVSCKHSGQCLKPCKDAGMRFGKCMNGKCDCTPK

SEQ ID NO: 250 - protein, 34 aa, Scorpio maurus palmatus
VSCTGSKDCYAPCRKQTGCPNAKCINKSCKCYGC

SEQ ID NO: 251 - protein, 36 aa, Mesobuthus tamulus
QFTDVDCSVSKECWSVCKDLFGVDRGKCMGKKCRCY

(19) United States
(12) Patent Application Publication
(10) Pub. No.: US 2016/0000869 A1
(43) Pub. Date: Jan. 7, 2016

(54) METHODS OF TREATING IPEX SYNDROME USING TOXIN-BASED PHARMACEUTICAL COMPOSITIONS

(71) Applicant: KINETA ONE, LLC, Seattle, WA (US)

(72) Inventors: Shawn P. Iadonato, Seattle, WA (US); Ernesto J. Munoz, Seattle, WA (US)

(21) Appl. No.: 14/772,611

(22) PCT Filed: Mar. 5, 2014
(86) PCT No.: PCT/US14/20771

§ 371 Date: Sep. 3, 2015

Related U.S. Application Data
(60) Provisional application No. 61/773,061, filed on Mar. 5, 2013.

(51) Int. Cl. A61K 38/17; G01N 33/50; A61K 47/26; C12Q 1/68; A61K 47/48; A61K 47/02

(52) U.S. Cl. CPC A61K 38/1767; A61K 47/48038; A61K 47/02; A61K 47/26; C12Q 1/6883; G01N 33/505; C12Q 2600/158; G01N 2333/57; G01N 2333/55; G01N 2333/5406; G01N 2333/5428; G01N 2333/54; G01N 2800/52; G01N 2800/24

(57) ABSTRACT

Disclosed herein are methods of treating immune dysregulation, polyendocrinopathy, enteropathy, X-linked syndrome (IPEX) comprising administering a toxin-based therapeutic peptide, such as an ShK-based peptide. The peptide can include an acid or amide at the C-terminus and/or the peptide can be attached to an organic or inorganic chemical entity that has an anionic charge.

### CROSS REFERENCE TO RELATED APPLICATIONS

This application is a national phase application based on International Patent Application No. PCT/US2014/020771, filed on Mar. 5, 2014, which claims priority to U.S. Provisional Patent Application No. 61/773,061, filed on Mar. 5, 2013, all of which are incorporated by reference herein in their entirety.

### FIELD OF THE DISCLOSURE

The methods disclosed herein relate generally to the use of toxin-based pharmaceutical compositions to treat IPEX syndrome. The toxin-based pharmaceutical compositions can include ShK-based peptides.

### BACKGROUND OF THE DISCLOSURE

Many immune-related human diseases and metabolic disorders are attributed to the action of T cells. Such immune-related diseases include, among others, immune dysregulation, polyendocrinopathy, enteropathy, X-linked syndrome (IPEX), and a genetic disorder that manifests in a variety of autoimmune pathologies. IPEX affects subjects from infancy. Depending on the severity, the condition can result in the occurrence of, for example, diabetes, eczema, thyroid dysfunction, enteropathy, and blood disorders. Subjects with IPEX rarely live beyond childhood, and only very occasionally into their teens or twenties.

### SUMMARY OF THE DISCLOSURE

Because IPEX is often manifested in different organs and tissues, it is often impractical to achieve successful treatment by focusing on one affected organ or tissue. Instead, there is a great need for methods to target underlying immune dysfunction and improve the health, longevity, and quality of life for individuals afflicted with IPEX.

Effector memory T cells (T_{EM}) initiate and contribute to many of the damaging processes associated with IPEX. These T_{EM} are highly dependent upon the Kv1.3 channel to sustain intracellular calcium levels required for activation, proliferation, and cytokine production.

One embodiment disclosed herein includes a method of treating IPEX comprising administering a toxin-based therapeutic peptide. In one embodiment, the peptide has the sequence of SEQ ID NO:1-251. In another embodiment, the peptide is an ShK-based peptide. In another embodiment, the peptide has the sequence of SEQ ID NO:1-224. In a further embodiment, the ShK-based peptide has the sequence of SEQ ID NO:1; SEQ ID NO:49; SEQ ID NO:22; SEQ ID NO: 217 (ShK-186); and/or SEQ ID NO: 210 (ShK-198).

In one method of treating IPEX, toxin-based therapeutic peptides, including in some embodiments, ShK-based peptides (referred to collectively herein as therapeutic peptides), can be attached to an organic or inorganic chemical entity that has an anionic charge. In another embodiment, the C-terminus can be an acid or an amide. In another embodiment, therapeutic peptides can be attached to an organic or inorganic chemical entity that has an anionic charge and the C-terminus is an acid or an amide.

In another method of treating IPEX, therapeutic peptides one or more chemical entities can be attached to the N terminus. In another embodiment, the chemical entity can be attached to the N-terminus of the therapeutic peptides through a linking molecule or linking group. In another embodiment, therapeutic peptides can have a chemical entity attached to the N-terminus by an aminoethyloxyethyloxy-acetyl linker.

Chemical entities can be selected from, without limitation, L-Pmp(OH₂); D-Pmp(OH₂); D-Pmp(OHEt); L-Pmp(Et₂); D-Pmp(Et₂); L-Tyr; L-Tyr(PO₃H₂); L-Phe(p-NH₂); L-Phe(p-CO₂H); L-Aspartate; D-Aspartate; L-Glutamate; and D-Glutamate.

Chemical entity/linker combinations can be selected from, without limitation, AEEAc-L-Pmp(OH₂); AEEAc-D-Pmp(OH₂); AEEAc-D-Pmp(OHEt); AEEAc-L-Pmp(Et₂); AEEAc-D-Pmp(Et₂); AEEAc-L-Tyr; AEEAc-L-Tyr(PO₃H₂); AEEAc-L-Phe(p-NH₂); AEEAc-L-Phe(p-CO₂H); AEEAc-L-Aspartate; AEEAc-D-Aspartate; AEEAc-L-Glutamate; and AEEAc-D-Glutamate.

In a method of treating IPEX, therapeutic peptides can be administered within a pharmaceutical composition. In another embodiment, the therapeutic peptides can be provided as pharmaceutically acceptable salts within the pharmaceutical compositions. In another embodiment, the pharmaceutically acceptable salt can be an acetate, such as potassium acetate or sodium acetate, and the pharmaceutical composition can be provided in an aqueous carrier.

In a method of treating IPEX, therapeutic peptides can be present at an amount from 0.01 mg/ml to 500 mg/ml. In additional embodiments, the therapeutic peptides can be provided in amount of 0.01, 0.1, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 150, 200, 250, 300, 350, 400, 450, or 500 mg/ml.

In a method of treating IPEX, therapeutic peptides can be obtained from a natural source. In another embodiment, therapeutic peptides can be synthetic. In another embodiment the therapeutic peptides can include a mixture of natural and synthetic peptides.

Embodiments described herein also include use of lyophilized pharmaceutical compositions produced beginning with a composition described herein. In one embodiment, the lyophilized pharmaceutical compositions comprise 8-12% acetate content by weight. In another embodiment, the lyophilized pharmaceutical compositions comprise 10-11% acetate content by weight.

In another embodiment of the use of lyophilized pharmaceutical compositions, the water content of the pharmaceutical compositions can be less than 5%. In another embodiment of the lyophilized pharmaceutical compositions, the water content can be less than 4.0%. In another embodiment of the lyophilized pharmaceutical compositions, the water content can be less than 3.5%.

In a method of treating IPEX, the pharmaceutical compositions can be formulated for subcutaneous administration. In another embodiment, the pharmaceutical compositions can be contained in a sterile syringe.

In another method of treating IPEX, a pharmaceutical composition can be administered wherein the pharmaceutical composition comprises a pharmaceutically acceptable salt of an ShK-based peptide having the sequence SEQ ID NO:1 and/or SEQ ID NO:49; 10 mM sodium phosphate; 150 mM NaCl; and Polysorbate 20 at 0.05 w/v %, wherein the ShK-based peptide is attached to an organic or inorganic chemical entity that has an anionic charge, the C-terminus is an acid or an amide, and the composition has a pH of 6.0.

In another method of treating IPEX, a pharmaceutical composition can be administered wherein the pharmaceutical composition comprises a pharmaceutically acceptable salt of an ShK-based peptide having the sequence of SEQ ID NO: 1; 10 mM sodium phosphate; 150 mM NaCl; and Polysorbate 20 at 0.05 w/v %, wherein the ShK-based peptide is attached to an organic or inorganic chemical entity that has an anionic charge, and the composition has a pH of 6.0.

In another method of treating IPEX, a pharmaceutical composition can be administered wherein the pharmaceutical composition comprises a pharmaceutically acceptable salt of an ShK-based peptide having the formula SEQ ID NO:217 (ShK-186); 10 mM sodium phosphate; 150 mM NaCl; and Polysorbate 20 at 0.05 w/v %, and wherein the composition has a pH of 6.0.

In another method of treating IPEX, a pharmaceutical composition can be administered wherein the pharmaceutical composition comprises an ShK-based peptide having the formula of SEQ ID NO:210 (ShK-198); 10 mM sodium phosphate; 150 mM NaCl; and Polysorbate 20 at 0.05 w/v %, and wherein the composition has a pH of 6.0.

In another method of treating IPEX, the pharmaceutical compositions can be administered daily, weekly, monthly, every two months, every three months, or every six months.

In another method of treating IPEX, the pharmaceutical composition can be administered subcutaneously.

In another method of treating IPEX, therapeutic peptides can be radiolabeled.

In another method of treating IPEX, therapeutic peptides can be labeled with ¹¹¹In.

In a further embodiment of the ¹¹¹In-labeled peptide, the therapeutic peptide is the ShK-based peptide, ShK-221 (SEQ ID NO:221).

The disclosure also provides methods of evaluating a subject to predict the outcome of treatment with a composition described herein, wherein a biological sample from the subject, such as peripheral blood mononuclear cells, is analyzed for T cell populations and levels of expression of Kv1.3, and wherein the ability of compositions disclosed herein to block the proinflammatory and proliferative potential of Memory T cells is measured. For example, a decrease in the activation, proliferation, cytokine production, perforin production or expression of Kv1.3 by Memory T cells in a subject (as compared to a previous measure from the same subject or as compared to a reference score derived from a population of individuals not affected by IPEX) can indicate that a treatment is effective. An increase or lack of a decrease in one or more of these parameters can indicate that a treatment is not effective.

In particular embodiments, the activation, proliferation, cytokine production, perforin production or expression of Kv1.3 by effector memory T cells (T_{EM}) is measured. A decrease in the activation, proliferation, cytokine production, perforin production or expression of Kv1.3 by T_{EM} in a subject (as compared to a previous measure from the same subject or as compared to a reference score derived from a population of individuals not affected by IPEX) can indicate that a treatment is effective. An increase or lack of a decrease in one or more of these parameters can indicate that a treatment is not effective.

Particular cytokines for measurement to assess the effectiveness of a treatment include IFN-γ; IL-2; IL-4; IL-10; IL-17 and IL-21.

Effectiveness of treatments can also be assessed by evaluating a number of clinically known parameters for conditions associated with IPEX described more fully elsewhere herein.

### DETAILED DESCRIPTION

Individuals affected by immune dysregulation, polyendocrinopathy, enteropathy, X-linked syndrome (IPEX) exhibit multiple autoimmune disorders, wherein the immune system attacks the body's tissues and organs. The systems most commonly affected by IPEX include the intestines, skin, and endocrine glands. Males are predominantly affected; the syndrome presents early in life and continues to be life-threatening in early childhood.

The cause of IPEX in about half of the subjects with this disorder has been identified as a mutation in the forkhead box P3 gene, referred to as FOXP3. The encoded protein is a transcription factor that binds to particular regions of the DNA and regulates gene expression. The protein is essential in the production and function of regulatory T cells.

FOXP3 gene mutations lead to reduced numbers or a complete absence of regulatory T cells (T_{REG}). In the absence of normal levels of T_{REG}, the body's immune responses can become uncontrolled and attack healthy tissues and organs. This disruption of T_{REG} function leads to the variety of autoimmune disorders characteristic of IPEX.

Because the described defect occurs at such a fundamental genetic level, treatment specific to the individual conditions (diabetes, eczema, and others discussed herein) do not fully alleviate or prevent the underlying disease. This explains why the prognosis for IPEX continues to be poor, and supports the urgent need for improved treatment. Currently, most afflicted individuals die within the first or second year of life, and even in mild cases, subjects rarely survive beyond the second or third decade of life. Death is usually due to metabolic dysfunction, severe malabsorption, or sepsis.

The intestines are particularly affected by IPEX and develop a condition referred to as enteropathy, wherein intestinal cells are destroyed by the immune system. The condition causes severe diarrhea, and is often an early symptom of IPEX as it manifests in the first few months of life. Enteropathy in turn leads to failure to thrive and cachexia (general wasting and weight loss).

Another system affected by IPEX is the skin. For example, inflammation, dermatitis, and eczema associated with IPEX cause abnormal patches of red, irritated skin. Afflicted individuals may exhibit other skin disorders with similar symptoms.

A third major effect of IPEX is polyendocrinopathy, which presents as multiple disorders of the endocrine glands. Some of the disorders are not unique to IPEX. For example, Type 1 diabetes mellitus is the most common endocrine disorder present in subjects with IPEX. Type 1 diabetes can develop within the first few months of life, preventing the body from regulating blood sugar. In addition to affecting the pancreas, IPEX can also involve autoimmune thyroid disease. Both hypothyroidism (underactive thyroid) and hyperthyroidism (overactive thyroid) have been observed.

Autoimmune blood disorders also occur in many subjects with IPEX, including anemia, thrombocytopenia, and neutropenia due to attack of the red cells, platelets, and white cells by the immune system.

IPEX may also involve other organs, such as the liver and kidneys. Most affected individuals therefore exhibit a variety of autoimmune phenomena including Coombs-positive anemia, immune thrombocytopenia, autoimmune neutropenia, hepatitis, and tubular nephropathy. Lymphadenopathy, splenomegaly, and alopecia have also been reported.

Subjects with IPEX are prone to serious and sometimes invasive infections, such as sepsis, meningitis, pneumonia, and osteomyelitis. More than 50% of individuals with IPEX are affected, with infections most commonly caused by Staphylococcus, Enterococcus, Cytomegalovirus, and Candida. The mechanisms underlying the infections are not fully understood, such as whether they relate to immunosuppressive therapy, whether they are characteristic of the disease itself, or a combination of factors. Regardless of the ultimate cause of the infections, the methods of treatment described herein can alleviate the infections by limiting autoimmune tissue damage and reducing the need for immunosuppressive drugs.

IPEX is attributed at least in part to the action of memory T cells. Two categories of memory T cells are known: central memory T cells (T_{CM}) and effector memory T cells (T_{EM}).

While IPEX is often a disorder of T_{REG} resulting from mutations in the FOXP3 gene, expanded populations of T_{EM} are also commonly observed. Upon activation, T_{EM} up-regulate Kv1.3 channels. Therefore the antigen-driven proliferation of T_{EM} is sensitive to Kv1.3 channel blockers.

T_{EM} that initiate and contribute to much of the damaging autoimmune processes observed with IPEX are highly dependent upon the Kv1.3 channel to sustain intracellular calcium levels required for activation, proliferation, and cytokine production. Thus, although the underlying genetic defect in T_{REG} cannot be easily corrected in an affected subject, T_{EM}, which in effect cause damage resulting from the T_{REG} defects, can be modulated.

In mouse models, complete loss of FOXP3 (Scurfy mouse) or reduced expression of FOXP3 results in T_{REG} deficiency and an increase in T_{EM}. To investigate the comparable mechanism in IPEX, decreased numbers of FOXP3-expressing T cells in peripheral blood can be determined by flow cytometry, as described in the Examples herein.

As noted above, a reduction in T_{REG} is manifested by an alteration in normal levels of T_{EM}, which exist as several subsets within the larger family of T cells. The T cell subsets are defined in part on the basis of their CD45 isoform expression: CD45RA, CD45RB, CD45RC, CD45RAB, CD45RAC, CD45RBC, CD45R0, and CD45R. CD45 is highly glycosylated.

The CD45 family members are products of a single complex gene. The gene contains thirty-four exons, and three exons of the primary transcripts are alternatively spliced to generate about eight different mature mRNAs which correlate with eight separate protein products. The three exons generate the RA, RB, and RC isoforms.

CD45R is the longest protein, and when isolated from T cells, migrates at 200 kDa. CD45RA is expressed on naïve T cells. CD45R0 is the shortest CD45 isoform and is expressed on memory T cells; CD45R0 lacks RA, RB and RC exons. This shortest isoform facilitates T cell activation.

The CD45 isoforms expressed on the various T cell subsets serve as markers for the status of the subject's immune system in terms of normal or abnormal numbers of naïve T cells and memory T cells.

In the present disclosure, T cell subsets are evaluated on the basis of their CD45RA, CD45R0, and CCR7 expression as naïve (CD45RA+/CD45R0−/CCR7+), T_{CM} (CD45RA−/CD45R0+/CCR7+) and T_{EM} (CD45RA−/CD45R0+/CCR7−) subsets.

Exemplary antibodies for measuring CD45 expression by the subsets of T cells include those provided by BD Biosciences, San Jose, Calif. 95131, including antibodies labeled for immunofluorescense. The labeled antibodies are suitable for performing flow cytometry as described in the Examples, and using a method such as that described in Zennaro, D. et al., Clin. Exp. Immunol. 167:120-128, 2012.

Levels of surface expression of Kv1.3 in the T cell populations can be assessed using an antibody that detects surface expression of the channel. An anti-potassium channel Kv1.3 (extracellular)-FITC antibody is available from Sigma-Aldrich, St. Louis, Mo.; antibodies are also available from LifeSpan Biosciences, Inc., Seattle, Wash.

Particular examples of toxin-based therapeutic peptides for use in the methods disclosed herein include toxin-based peptides, including ShK-based peptides, that target voltage gated channels. Exemplary voltage gated channels include Kv1.1, Kv1.2, Kv1.3, Kv1.4, Kv1.5, Kv1.6, Kv1.7, Kv2.1, Kv3.1, Kv3.2, Kv11.1, Kc1.1, Kc2.1, Kc3.1, Nav1.2, Nav1.4, and Cav1.2.

Toxin peptides are produced by a variety of organisms and have evolved to target ion channels and receptors. Native toxin peptides from snakes, scorpions, spiders, bees, snails, and sea anemone are typically 10-80 amino acids in length and contain 2 to 5 disulfide bridges that create compact molecular structures. These peptides appear to have evolved from a small number of structural frameworks. The peptides cluster into families of folding patterns that are conserved through cysteine/disulfide loop structures to maintain a three dimensional structure that contributes to potency, stability, and selectivity (Pennington, et al., Biochemistry 38:14549-58,1999; Tudor, et al., Eur. J. Biochem. 251:133-41, 1998; and Jaravine et al., Biochemistry 36: 1223-32, 1997). As used herein, “toxin-based therapeutic peptides” consist, consist essentially of or consist of any synthetic or naturally-known toxin peptides and derivatives and analogs thereof.

As used herein, “derivatives” of peptides include peptides having one or more amino acid substitutions as compared to a naturally-occurring peptide sequence. The substitution can be a conservative or a non-conservative substitution. Derivatives of peptides also include additions of amino acids, deletions of amino acids, modifications of amino acids and stop positions added or inserted into a naturally-occurring peptide sequence.

As used herein, “modifications” of amino acids include replacing an amino acid in a sequence with a non-amino acid component or conjugating or otherwise associating a functional group to an amino acid. The modified amino acid can be within the sequence or at the terminal end of a sequence.

As used herein, “analogs” include amino acid sequences having one more L-amino acids replaced with D-amino acids. The D-amino acid can be the same amino acid type as that found in the natural sequence or can be a different amino acid. Accordingly, some analogs are also derivatives.

As used herein, a “conservative substitution” involves a substitution of one amino acid for another found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gin); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

Sequence information provided by public database can be used to identify sequences of therapeutic peptides not otherwise explicitly disclosed herein as well as nucleic acid sequences encoding the therapeutic peptides.

Particular exemplary toxin-based therapeutic peptides for use in the methods disclosed herein can comprise, consist essentially of, or consist of those listed in Table 1, and as shown in the sequence listing as SEQ ID NO:225-251. In various embodiments, a method of treating IPEX comprises administering a toxin-based therapeutic peptide of Table 1 (SEQ ID NO:225-251). In various embodiments, the toxin-based therapeutic peptides of Table 1 (SEQ ID NO:225-251) can be used in the production of a medicament to treat IPEX.

TABLE 1
Exemplary Toxin-Based Therapeutic Peptides

| | Short- | |
|---|---|---|
| | hand | SEQ |
| | desig- | ID |
| Sequence/structure | nation | NO: |
| LVKCRGTSDCGRPCQQQTGCPNSKCINRMCKCYGC | Pi1 | 225 |
| TISCTNPKQCYPHCKKETGYPNAKCMNRKCKCFGR | Pi2 | 226 |
| TISCTNEKQCYPHCKKETGYPNAKCMNRKCKCFGR | Pi3 | 227 |
| IEAIRCGGSRDCYRPCQKRTGCPNAKCINKTCKCYGCS | Pi4 | 228 |
| ASCRTPKDCADPCRKETGCPYGKCMNRKCKCNRC | HsTx1 | 229 |
| GVPINVSCTGSPQCIKPCKDAGMRFGKCMNRKCHCTPK | AgTx2 | 230 |
| GVPINVKCTGSPQCLKPCKDAGMRFGKCINGKCHCTPK | AgTx1 | 231 |
| GVIINVKCKISRQCLEPCKKAGMRFGKCMNGKCHCTPK | OSK1 | 232 |
| ZKECTGPQHCTNFCRKNKCTHGKCMNRKCKCFNCK | Anuroc- | 232 |
| | toxin | |
| TIINVKCTSPKQCSKPCKELYGSSAGAKCMNGKCKCYNN | NTx | 234 |
| TVIDVKCTSPKQCLPPCKAQFGIRAGAKCMNGKCKCYPH | HgTx1 | 235 |
| QFTNVSCTTSKECWSVCQRLHNTSRGKCMNKKCRCYS | ChTx | 236 |
| VFINAKCRGSPECLPKCKEAIGKAAGKCMNGKCKCYP | Titys- | 237 |
| | toxin-Ka | |
| VCRDWFKETACRHAKSLGNCRTSQKYRANCAKTCELC | BgK | 238 |
| VGINVKCKHSGQCLKPCKDAGMRFGKCINGKCDCTPKG | BmKTx | 239 |
| QFTDVKCTGSKQCWPVCKQMFGKPNGKCMNGKCRCYS | BmTx1 | 240 |
| VFINVKCRGSKECLPACKAAVGKAAGKCMNGKCKCYP | Tc30 | 241 |
| TGPQTTCQAAMCEAGCKGLGKSMESCQGDTCKCKA | Tc32 | 242 |
| AAAISCVGSPECPPKCRAQGCKNGKCMNRKCKCYYC- | Vm24 | 243 |
| amide | | |
| RTCKDLIPVSECTDIRCRTSMKYRLNLCRKTCGSC | HmK | 244 |
| GCKDNFSANTCKHVKANNNCGSQKYATNCAKTCGKC | Aek | 245 |
| ACKDNFAAATCKHVKENKNCGSQKYATNCAKTCGKC | AsKS | 246 |
| TIINVKCTSPKQCLPPCKAQFGQSAGAKCMNGKCKCYPH | MgTx | 247 |
| GVEINVKCSGSPQCLKPCKDAGMRFGKCMNRKCHCTPK | KTx1 | 248 |
| VRIPVSCKHSGQCLKPCKDAGMRFGKCMNGKCDCTPK | KTx2 | 249 |
| VSCTGSKDCYAPCRKQTGCPNAKCINKSCKCYGC | MTx | 250 |
| QFTDVDCSVSKECWSVCKDLFGVDRGKCMGKKCRCY | IbTx | 251 |

“ShK” peptides are a subtype of toxin peptides that can also be used in the methods described herein. ShK peptides were originally isolated from the Caribbean sea anemone Stichodactyla helianthus. ShK peptides serve as inhibitors of, without limitation, Kv1.3 channels. By inhibiting Kv1.3 channels, ShK can suppress activation, proliferation and/or cytokine production of or by T_{EM}, in certain embodiments, at picomolar concentrations.

As used herein, an “inhibitor” is any therapeutic peptide that decreases or eliminates a biological activity that normally results based on the interaction of a compound with a receptor including, without limitation, biosynthetic and/or catalytic activity, receptor or signal transduction pathway activity, gene transcription or translation, cellular protein transport, etc.

A native ShK peptide is described in, for example, Pennington, et al., Int. J. Pept. Protein Res. 46:354-358,1995. Exemplary ShK structures that are within the scope of the present disclosure are also published in, without limitation, Beeton, et al., Mol. Pharmacol., 67:1369-1381, 2005; U.S. Publication No. 2008/0221024; PCT Publication No. WO/2012/170392; and in U.S. Pat. Nos. 8,080,523 and 8,440,621

As used herein, As used herein, “ShK-based peptides” consist, consist essentially of or consist of any synthetic or naturally-known ShK peptides and derivatives and analogs thereof. ShK-based peptides include the peptides of SEQ ID NO:1 and/or SEQ ID NO:2-224 to which an organic or inorganic chemical entity that has an anionic charge is attached via an aminoethyloxyethyloxy-acetyl linker. For use in the disclosed methods, ShK-based peptides can be provided within a pharmaceutical composition as a mixture of naturally occurring and synthetic ShK-based peptides.

Exemplary ShK-based peptides for use in the methods disclosed herein are shown in SEQ ID NO:1, SEQ ID NO:49, SEQ ID NO:210, SEQ ID NO:217, and SEQ ID NO:221. In additional embodiments, the ShK-based peptides for use in the methods described herein are shown in SEQ ID NO:1-224.

An example of an ShK-based peptide is ShK-186 (SEQ ID NO: 217). ShK-186 (and all other ShK-based peptides and toxin-based therapeutic peptides disclosed herein) can be modified by the N-terminal attachment of aminoethyloxyethyloxyacetic acid (referred to as AEEAc), and/or an amide attachment at the C-terminal (for example, SEQ ID NO:217). As used herein, the term AEEAc can interchangeably refer to aminoethyloxyethyloxyacetic acid and Fmoc-aminoethyloxyethyloxyacetic acid when being used to describe the linker during the formation process. When being used to refer to the linker in specific peptides in their final state, the term refers to aminoethyloxyethyloxyacetic acid.

Another example of an ShK-based peptide is a DOTA-conjugate of ShK-186 (referred to as ShK-221). “DOTA” refers to 1,4,7,10-tetraazacyclododecane-1,4,7,10-tetraacetic acid which can be attached to the N-terminus of the therapeutic peptides disclosed herein via aminohexanoic acid. DOTA conjugation provides a site for chelating metal atoms such as Indium or Gadolinium. Other molecules that can be conjugated to therapeutic peptides disclosed herein include, without limitation, diethylene triamine pentaacetic acid (DTPA), Nitrilotriacetic acid (NTA), Ethylenediaminetetraacetic acid (EDTA), Iminodiacetic acid (IDA), ethylene glycol tetraacetic acid (EGTA), 1,2-bis(o-aminophenoxy)ethane-N,N,N′,N′-tetraacetic acid (BAPTA), 1,4,7-triazacyclononane-N,N′,N″-triacetic acid (NOTA) and related molecules. A further example is a substitution or modification of SEQ ID NO:1 wherein the amino acid at position 21 is Norleucine and/or the amino acid at position 22 is replaced with diaminopropionic acid.

Particular exemplary ShK-based peptides for use with the pharmaceutical compositions disclosed herein can comprise, consist essentially of or consist of those listed in Table 2, and as shown in the sequence listing as SEQ ID NO:1-224. In various embodiments, a method of treating IPEX comprises administering a ShK-based peptide of Table 2 (SEQ ID NO:1-224). In various embodiments, the ShK-based peptides of Table 2 (SEQ ID NO:1-224) can be used in the production of a medicament to treat IPEX.

TABLE 2
Exemplary ShK-Based Peptides

| | | SEQ |
|---|---|---|
| | | ID |
| Sequence/structure | Shorthand ID | NO: |
| RSCIDTIPKSRCTAFQCKHSMKYRLSFCRKTCGTC | ShK | 1 |
| RSCIDTIPKSRCTAFQSKHSMKYRLSFCRKTSGTC | ShK-S17/S32 | 2 |
| RSSIDTIPKSRCTAFQCKHSMKYRLSFCRKTCGTS | ShK-S3/S35 | 3 |
| SSCIDTIPKSRCTAFQCKHSMKYRLSFCRKTCGTC | ShK-S1 | 4 |
| (N-acetylR)SCIDTIPKSRCTAFQCKHSMKYRLSFCRKT | ShK-N-acetylarg1 | 5 |
| CGTC | | |
| SCIDTIPKSRCTAFQCKHSMKYRLSFCRKTCGTC | ShK-d1 | 6 |
| CIDTIPKSRCTAFQCKHSMKYRLSFCRKTCGTC | ShK-d2 | 7 |
| ASCIDTIPKSRCTAFQCKHSMKYRLSFCRKTCGTC | ShK-A1 | 8 |
| QCIDTIPKSRCTAFQCKHSMKYRLSFCRKTCGTC | ShK-Q2 d1 | 9 |
| ACIDTIPKSRCTAFQCKHSMKYRLSFCRKTCGTC | ShK-A2 d1 | 10 |
| TCIDTIPKSRCTAFQCKHSMKYRLSFCRKTCGTC | ShK-T2 d1 | 11 |
| RQCIDTIPKSRCTAFQCKHSMKYRLSFCRKTCGTC | ShK-Q2 | 12 |
| RACIDTIPKSRCTAFQCKHSMKYRLSFCRKTCGTC | ShK-A2 | 13 |
| RTCIDTIPKSRCTAFQCKHSMKYRLSFCRKTCGTC | ShK-T2 | 14 |
| AQCIDTIPKSRCTAFQCKHSMKYRLSFCRKTCGTC | ShK-Q2 | 15 |
| AACIDTIPKSRCTAFQCKHSMKYRLSFCRKTCGTC | ShK-A1/A2 | 16 |
| ATCIDTIPKSRCTAFQCKHSMKYRLSFCRKTCGTC | ShK-A1/T2 | 17 |
| RSCADTIPKSRCTAFQCKHSMKYRLSFCRKTCGTC | ShK-A1/A4 | 18 |
| RSCADTIPKSRCTAAQCKHSMKYRLSFCRKTCGTC | ShK-A4/A15 | 19 |
| RSCADTIPKSRCTAAQCKHSMKYRASFCRKTCGTC | ShK-A4/A15/A25 | 20 |
| RSCIDAIPKSRCTAFQCKHSMKYRLSFCRKTCGTC | ShK-A6 | 21 |
| RSCIDTIPKSRCTAFQCKHSMKYRLSFCRKTCGTC- | ShK-T6 | 22 |
| amide | | |
| RSCIDYIPKSRCTAFQCKHSMKYRLSFCRKTCGTC | ShK-Y6 | 23 |
| RSCIDLIPKSRCTAFQCKHSMKYRLSFCRKTCGTC | ShK-L6 | 24 |
| RSCIDTAPKSRCTAFQCKHSMKYRLSFCRKTCGTC | ShK-A7 | 25 |
| RSCADTIPKSRCTAFQCKHSMKYRLSFCRKTCGTC | ShK-A4 | 26 |
| RSCIDTIAKSRCTAFQCKHSMKYRLSFCRKTCGTC | ShK-A8 | 27 |
| RSCIDTIPASRCTAFQCKHSMKYRLSFCRKTCGTC | ShK-A9 | 28 |
| RSCIDTIPESRCTAFQCKHSMKYRLSFCRKTCGTC | ShK-E9 | 29 |
| RSCIDTIPQSRCTAFQCKHSMKYRLSFCRKTCGTC | ShK-Q9 | 30 |
| RSCIDTIPKARCTAFQCKHSMKYRLSFCRKTCGTC | ShK-A10 | 31 |
| RSCIDTIPKSACTAFQCKHSMKYRLSFCRKTCGTC | ShK-A11 | 32 |
| RSCIDTIPKSECTAFQCKHSMKYRLSFCRKTCGTC | ShK-E11 | 33 |
| RSCIDTIPKSQCTAFQCKHSMKYRLSFCRKTCGTC | ShK-Q11 | 34 |
| RSCIDTIPKSRCAAFQCKHSMKYRLSFCRKTCGTC | ShK-A13 | 35 |
| RSCIDTIPKSRCTAAQCKHSMKYRLSFCRKTCGTC | ShK-A15 | 36 |
| RSCIDTIPKSRCTAWQCKHSMKYRLSFCRKTCGTC | ShK-W15 | 37 |
| RSCIDTIPKSRCTA[X(s1)]QCKHSMKYRLSFCRKTCGTC | ShK-X15 | 38 |
| RSCIDTIPKSRCTAAQCKHSMKYRASFCRKTCGTC | ShK-A15/A25 | 39 |
| RSCIDTIPKSRCTAFACKHSMKYRLSFCRKTCGTC | ShK-A16 | 40 |
| RSCIDTIPKSRCTAFECKHSMKYRLSFCRKTCGTC | ShK-E16 | 41 |
| RSCIDTIPKSRCTAFQCAHSMKYRLSFCRKTCGTC | ShK-A18 | 42 |
| RSCIDTIPKSRCTAFQCEHSMKYRLSFCRKTCGTC | ShK-E18 | 43 |
| RSCIDTIPKSRCTAFQCKASMKYRLSFCRKTCGTC | ShK-A19 | 44 |
| RSCIDTIPKSRCTAFQCKKSMKYRLSFCRKTCGTC | ShK-K19 | 45 |
| RSCIDTIPKSRCTAFQCKHAMKYRLSFCRKTCGTC | ShK-A20 | 46 |
| RSCIDTIPKSRCTAFQCKHSAKYRLSFCRKTCGTC | ShK-A21 | 47 |
| RSCIDTIPKSRCTAFQCKHS[X(s2)]KYRLSFCRKTCGTC | ShK-X21 | 48 |
| RSCIDTIPKSRCTAFQCKHS(Nle)KYRLSFCRKTCGTC | ShK-Nle21 | 49 |
| RSCIDTIPKSRCTAFQCKHSMAYRLSFCRKTCGTC | ShK-A22 | 50 |
| RSCIDTIPKSRCTAFQCKHSMEYRLSFCRKTCGTC | ShK-E22 | 51 |
| RSCIDTIPKSRCTAFQCKHSMRYRLSFCRKTCGTC | ShK-R22 | 52 |
| RSCIDTIPKSRCTAFQCKHSM[X(s3)]YRLSFCRKTCGTC | ShK-X22 | 53 |
| RSCIDTIPKSRCTAFQCKHSM(Nle)YRLSFCRKTCGTC | ShK-Nle22 | 54 |
| RSCIDTIPKSRCTAFQCKHSM(Orn)YRLSFCRKTCGTC | ShK-Orn22 | 55 |
| RSCIDTIPKSRCTAFQCKHSM(Homocit)YRLSFCRKTCGTC | ShK-Homocit22 | 56 |
| RSCIDTIPKSRCTAFQCKHSM(Dap)YRLSFCRKTCGTC | ShK-diamino-propionic22 | 57 |
| RSCIDTIPKSRCTAFQCKHSMKARLSFCRKTCGTC | ShK-A23 | 58 |
| RSCIDTIPKSRCTAFQCKHSMKSRLSFCRKTCGTC | ShK-S23 | 59 |
| RSCIDTIPKSRCTAFQCKHSMKFRLSFCRKTCGTC | ShK-F23 | 60 |
| RSCIDTIPKSRCTAFQCKHSMK[X(s4)]RLSFCRKTCGTC | ShK-X23 | 61 |
| RSCIDTIPKSRCTAFQCKHSMK(NitroF)RLSFCRKTCGTC | ShK-Nitrophe23 | 62 |
| RSCIDTIPKSRCTAFQCKHSMK(AminoF)RLSFCRKTCGTCC | ShK-Aminophe23 | 63 |
| RSCIDTIPKSRCTAFQCKHSMK(BenzylF)RLSFCRKTCGTC | ShK-Benzylphe23 | 64 |
| RSCIDTIPKSRCTAFQCKHSMKYALSFCRKTCGTC | ShK-A24 | 65 |
| RSCIDTIPKSRCTAFQCKHSMKYELSFCRKTCGTC | ShK-E24 | 66 |
| RSCIDTIPKSRCTAFQCKHSMKYRASFCRKTCGTC | ShK-A25 | 67 |
| RSCIDTIPKSRCTAFQCKHSMKYRLAFCRKTCGTC | ShK-A26 | 68 |
| RSCIDTIPKSRCTAFQCKHSMKYRLSACRKTCGTC | ShK-A27 | 69 |
| RSCIDTIPKSRCTAFQCKHSMKYRLS[X(s27)]CRKTCGTC | ShK-X27 | 70 |
| RSCIDTIPKSRCTAFQCKHSMKYRLSFCAKTCGTC | ShK-A29 | 71 |
| RSCIDTIPKSRCTAFQCKHSMKYRLSFCRATCGTC | ShK-A30 | 72 |
| RSCIDTIPKSRCTAFQCKHSMKYRLSFCRKACGTC | ShK-A31 | 73 |
| RSCIDTIPKSRCTAFQCKHSMKYRLSFCRKTCGAC | ShK-A34 | 74 |
| SCADTIPKSRCTAFQCKHSMKYRLSFCRKTCGTC | ShK-A4d1 | 75 |
| SCADTIPKSRCTAAQCKHSMKYRLSFCRKTCGTC | ShK-A4/A15d1 | 76 |
| SCADTIPKSRCTAAQCKHSMKYRASFCRKTCGTC | ShK-A4/A15/A25d1 | 77 |
| SCIDAIPKSRCTAFQCKHSMKYRLSFCRKTCGTC | ShK-A6d1 | 78 |
| SCIDTAPKSRCTAFQCKHSMKYRLSFCRKTCGTC | ShK-A7d1 | 79 |
| SCIDTIAKSRCTAFQCKHSMKYRLSFCRKTCGTC | ShK-A8d1 | 80 |
| SCIDTIPASRCTAFQCKHSMKYRLSFCRKTCGTC | ShK-A9d1 | 81 |
| SCIDTIPESRCTAFQCKHSMKYRLSFCRKTCGTC | ShK-E9d1 | 82 |
| SCIDTIPQSRCTAFQCKHSMKYRLSFCRKTCGTC | ShK-Q9d1 | 83 |
| SCIDTIPKARCTAFQCKHSMKYRLSFCRKTCGTC | ShK-A10d1 | 84 |
| SCIDTIPKSACTAFQCKHSMKYRLSFCRKTCGTC | ShK-A11d1 | 85 |
| SCIDTIPKSECTAFQCKHSMKYRLSFCRKTCGTC | ShK-E11d1 | 86 |
| SCIDTIPKSQCTAFQCKHSMKYRLSFCRKTCGTC | ShK-Q11d1 | 87 |
| SCIDTIPKSRCAAFQCKHSMKYRLSFCRKTCGTC | ShK-A13d1 | 88 |
| SCIDTIPKSRCTAAQCKHSMKYRLSFCRKTCGTC | ShK-A15d1 | 89 |
| SCIDTIPKSRCTAWQCKHSMKYRLSFCRKTCGTC | ShK-W15d1 | 90 |
| SCIDTIPKSRCTA[X(s15)]QCKHSMKYRLSFCRKTCGTC | ShK-X15d1 | 91 |
| SCIDTIPKSRCTAAQCKHSMKYRASFCRKTCGTC | ShK-A15/A25d1 | 92 |
| SCIDTIPKSRCTAFACKHSMKYRLSFCRKTCGTC | ShK-A16d1 | 93 |
| SCIDTIPKSRCTAFECKHSMKYRLSFCRKTCGTC | ShK-E16d1 | 94 |
| SCIDTIPKSRCTAFQCAHSMKYRLSFCRKTCGTC | ShK-A18d1 | 95 |
| SCIDTIPKSRCTAFQCEHSMKYRLSFCRKTCGTC | ShK-E18d1 | 96 |
| SCIDTIPKSRCTAFQCKASMKYRLSFCRKTCGTC | ShK-A19d1 | 97 |
| SCIDTIPKSRCTAFQCKKSMKYRLSFCRKTCGTC | ShK-K19d1 | 98 |
| SCIDTIPKSRCTAFQCKHAMKYRLSFCRKTCGTC | ShK-A20d1 | 99 |
| SCIDTIPKSRCTAFQCKHSAKYRLSFCRKTCGTC | ShK-A21d1 | 100 |
| SCIDTIPKSRCTAFQCKHS[X(s2)]KYRLSFCRKTCGTC | ShK-X21d1 | 101 |
| SCIDTIPKSRCTAFQCKHS(NIe)KYRLSFCRKTCGTC | ShK-Nle21d1 | 102 |
| SCIDTIPKSRCTAFQCKHSMAYRLSFCRKTCGTC | ShK-A22d1 | 103 |
| SCIDTIPKSRCTAFQCKHSMEYRLSFCRKTCGTC | ShK-E22d1 | 104 |
| SCIDTIPKSRCTAFQCKHSMRYRLSFCRKTCGTC | ShK-R22d1 | 105 |
| SCIDTIPKSRCTAFQCKHSM[X(s3)]YRLSFCRKTCGTC | ShK-X22d1 | 106 |
| SCIDTIPKSRCTAFQCKHSM(Nle)YRLSFCRKTCGTC | ShK-Nle22d1 | 107 |
| SCIDTIPKSRCTAFQCKHSM(Orn)YRLSFCRKTCGTC | ShK-Orn22d1 | 108 |
| SCIDTIPKSRCTAFQCKHSM(Homocit)YRLSFCRKTCGTC | ShK-Homocit22 d1 | 109 |
| SCIDTIPKSRCTAFQCKHSM(Dap)YRLSFCRKTCGTC | ShK-Dap22d1 | 110 |
| SCIDTIPKSRCTAFQCKHSMKARLSFCRKTCGTC | ShK-A23d1 | 111 |
| SCIDTIPKSRCTAFQCKHSMKSRLSFCRKTCGTC | ShK-S23d1 | 112 |
| SCIDTIPKSRCTAFQCKHSMKFRLSFCRKTCGTC | ShK-F23d1 | 113 |
| SCIDTIPKSRCTAFQCKHSMK[X(s4)]RLSFCRKTCGTC | ShK-X23d1 | 114 |
| SCIDTIPKSRCTAFQCKHSMK(NitroF)RLSFCRKTCGTC | ShK-Nitrophe23d1 | 115 |
| SCIDTIPKSRCTAFQCKHSMK(AminoF)RLSFCRKTCGTC | ShK-Aminophe23d1 | 116 |
| SCIDTIPKSRCTAFQCKHSMK(BenzylF)RLSFCRKTCGT | ShK-Benzylphe23d1 | 117 |
| SCIDTIPKSRCTAFQCKHSMKYALSFCRKTCGTC | ShK-A24d1 | 118 |
| SCIDTIPKSRCTAFQCKHSMKYELSFCRKTCGTC | ShK-E24d1 | 119 |
| SCIDTIPKSRCTAFQCKHSMKYRASFCRKTCGTC | ShK-A25d1 | 120 |
| SCIDTIPKSRCTAFQCKHSMKYRLAFCRKTCGTC | ShK-A26d1 | 121 |
| SCIDTIPKSRCTAFQCKHSMKYRLSACRKTCGTC | ShK-A27d1 | 122 |
| SCIDTIPKSRCTAFQCKHSMKYRLS[X(s5)]CRKTCGTC | ShK-X27d1 | 123 |
| SCIDTIPKSRCTAFQCKHSMKYRLSFCAKTCGTC | ShK-A29d1 | 124 |
| SCIDTIPKSRCTAFQCKHSMKYRLSFCRATCGTC | ShK-A30d1 | 125 |
| SCIDTIPKSRCTAFQCKHSMKYRLSFCRKACGTC | ShK-A31d1 | 126 |
| SCIDTIPKSRCTAFQCKHSMKYRLSFCRKTCGAC | ShK-A34d1 | 127 |
| YSCIDTIPKSRCTAFQCKHSMKYRLSFCRKTCGTC | ShK-Y1 | 128 |
| KSCIDTIPKSRCTAFQCKHSMKYRLSFCRKTCGTC | ShK-K1 | 129 |
| HSCIDTIPKSRCTAFQCKHSMKYRLSFCRKTCGTC | ShK-H1 | 130 |
| QSCIDTIPKSRCTAFQCKHSMKYRLSFCRKTCGTC | ShK-Q1 | 131 |
| PPRSCIDTIPKSRCTAFQCKHSMKYRLSFCRKTCGTC | PP-ShK | 132 |
| MRSCIDTIPKSRCTAFQCKHSMKYRLSFCRKTCGTC | M-ShK | 133 |
| GRSCIDTIPKSRCTAFQCKHSMKYRLSFCRKTCGTC | G-ShK | 134 |
| YSCIDTIPKSRCTAFQCKHSMAYRLSFCRKTCGTC | ShK-Y1/A22 | 135 |
| KSCIDTIPKSRCTAFQCKHSMAYRLSFCRKTCGTC | ShK-K1/A22 | 136 |
| HSCIDTIPKSRCTAFQCKHSMAYRLSFCRKTCGTC | ShK-H1/A22 | 137 |
| QSCIDTIPKSRCTAFQCKHSMAYRLSFCRKTCGTC | ShK-Q1/A22 | 138 |
| PPRSCIDTIPKSRCTAFQCKHSMAYRLSFCRKTCGTC | PP-ShK-A22 | 139 |
| MRSCIDTIPKSRCTAFQCKHSMAYRLSFCRKTCGTC | M-ShK-A22 | 140 |
| GRSCIDTIPKSRCTAFQCKHSMAYRLSFCRKTCGTC | G-ShK-A22 | 141 |
| RSCIDTIPASRCTAFQCKHSMAYRLSFCRKTCGTC | ShK-A9/A22 | 142 |
| SCIDTIPASRCTAFQCKHSMAYRLSFCRKTCGTC | ShK-A9/A22d1 | 143 |
| RSCIDTIPVSRCTAFQCKHSMKYRLSFCRKTCGTC | ShK-V9 | 144 |
| RSCIDTIPVSRCTAFQCKHSMAYRLSFCRKTCGTC | ShK-V9/A22 | 145 |
| SCIDTIPVSRCTAFQCKHSMKYRLSFCRKTCGTC | ShK-V9d1 | 146 |
| SCIDTIPVSRCTAFQCKHSMAYRLSFCRKTCGTC | ShK-V9/A22d1 | 147 |
| RSCIDTIPESRCTAFQCKHSMAYRLSFCRKTCGTC | ShK-E9/A22 | 148 |
| SCIDTIPESRCTAFQCKHSMAYRLSFCRKTCGTC | ShK-E9/A22d1 | 149 |
| RSCIDTIPKSACTAFQCKHSMAYRLSFCRKTCGTC | ShK-A11/A22 | 150 |
| SCIDTIPKSACTAFQCKHSMAYRLSFCRKTCGTC | ShK-A11/A22d1 | 151 |
| RSCIDTIPKSECTAFQCKHSMAYRLSFCRKTCGTC | ShK-E11/A22 | 152 |
| SCIDTIPKSECTAFQCKHSMAYRLSFCRKTCGTC | ShK-E11/A22d1 | 153 |
| RSCIDTIPKSRCTDFQCKHSMKYRLSFCRKTCGTC | ShK-D14 | 154 |
| RSCIDTIPKSRCTDFQCKHSMAYRLSFCRKTCGTC | ShK-D14/A22 | 155 |
| SCIDTIPKSRCTDFQCKHSMKYRLSFCRKTCGTC | ShK-D14d1 | 156 |
| SCIDTIPKSRCTDFQCKHSMAYRLSFCRKTCGTC | ShK-D14/A22d1 | 157 |
| RSCIDTIPKSRCTAAQCKHSMAYRLSFCRKTCGTC | ShK-A15/A22 | 158 |
| SCIDTIPKSRCTAAQCKHSMAYRLSFCRKTCGTC | ShK-A15/A22d1 | 159 |
| RSCIDTIPKSRCTAIQCKHSMKYRLSFCRKTCGTC | ShK-I15 | 160 |
| RSCIDTIPKSRCTAIQCKHSMAYRLSFCRKTCGTC | ShK-I15/A22 | 161 |
| SCIDTIPKSRCTAIQCKHSMKYRLSFCRKTCGTC | ShK-I15d1 | 162 |
| SCIDTIPKSRCTAIQCKHSMAYRLSFCRKTCGTC | ShK-I15/A22d1 | 163 |
| RSCIDTIPKSRCTAVQCKHSMKYRLSFCRKTCGTC | ShK-V15 | 164 |
| RSCIDTIPKSRCTAVQCKHSMAYRLSFCRKTCGTC | ShK-V15/A22 | 165 |
| SCIDTIPKSRCTAVQCKHSMKYRLSFCRKTCGTC | ShK-V15d1 | 166 |
| SCIDTIPKSRCTAVQCKHSMAYRLSFCRKTCGTC | ShK-V15/A22d1 | 167 |
| RSCIDTIPKSRCTAFRCKHSMKYRLSFCRKTCGTC | ShK-R16 | 168 |
| RSCIDTIPKSRCTAFRCKHSMAYRLSFCRKTCGTC | ShK-R16/A22 | 169 |
| SCIDTIPKSRCTAFRCKHSMKYRLSFCRKTCGTC | ShK-R16d1 | 170 |
| SCIDTIPKSRCTAFRCKHSMAYRLSFCRKTCGTC | ShK-R16/A22d1 | 171 |
| RSCIDTIPKSRCTAFKCKHSMKYRLSFCRKTCGTC | ShK-K16 | 172 |
| RSCIDTIPKSRCTAFKCKHSMAYRLSFCRKTCGTC | ShK-K16/A22 | 173 |
| SCIDTIPKSRCTAFKCKHSMKYRLSFCRKTCGTC | ShK-K16d1 | 174 |
| SCIDTIPKSRCTAFKCKHSMAYRLSFCRKTCGTC | ShK-K16/A22d1 | 175 |
| RSCIDTIPASECTAFQCKHSMKYRLSFCRKTCGTC | ShK-A9/E11 | 176 |
| RSCIDTIPASECTAFQCKHSMAYRLSFCRKTCGTC | ShK-A9/E11/A22 | 177 |
| SCIDTIPASECTAFQCKHSMKYRLSFCRKTCGTC | ShK-A9/E11d1 | 178 |
| SCIDTIPASECTAFQCKHSMAYRLSFCRKTCGTC | ShK-A9/E11/A22d1 | 179 |
| RSCIDTIPVSECTAFQCKHSMKYRLSFCRKTCGTC | ShK-V9/E11 | 180 |
| RSCIDTIPVSECTAFQCKHSMAYRLSFCRKTCGTC | ShK-V9/E11/A22 | 181 |
| SCIDTIPVSECTAFQCKHSMKYRLSFCRKTCGTC | ShK-V9/E11d1 | 182 |
| SCIDTIPVSECTAFQCKHSMAYRLSFCRKTCGTC | ShK-V9/E11/A22d1 | 183 |
| RSCIDTIPVSACTAFQCKHSMKYRLSFCRKTCGTC | ShK-V9/A11 | 184 |
| RSCIDTIPVSACTAFQCKHSMAYRLSFCRKTCGTC | ShK-V9/A11/A22 | 185 |
| SCIDTIPVSACTAFQCKHSMKYRLSFCRKTCGTC | ShK-V9/A11d1 | 186 |
| SCIDTIPVSACTAFQCKHSMAYRLSFCRKTCGTC | ShK-V9/A11/A22d1 | 187 |
| RSCIDTIPASACTAFQCKHSMKYRLSFCRKTCGTC | ShK-A9/A11 | 188 |
| RSCIDTIPASACTAFQCKHSMAYRLSFCRKTCGTC | ShK-A9/A11/A22 | 189 |
| SCIDTIPASACTAFQCKHSMKYRLSFCRKTCGTC | ShK-A9/A11d1 | 190 |
| SCIDTIPASACTAFQCKHSMAYRLSFCRKTCGTC | ShK-A9/A11/A22d1 | 191 |
| RSCIDTIPKSECTDIRCKHSMKYRLSFCRKTCGTC | ShK-E11/D14/I15/R16 | 192 |
| RSCIDTIPKSECTDIRCKHSMAYRLSFCRKTCGTC | ShK-E11/D14/I15/ | 193 |
| | R16/A22 | |
| SCIDTIPKSECTDIRCKHSMKYRLSFCRKTCGTC | ShK-Ell/D14/I15/R16d1 | 194 |
| SCIDTIPKSECTDIRCKHSMAYRLSFCRKTCGTC | ShK-E11/D14/I15/ | 195 |
| | R16/A22d1 | |
| RSCIDTIPVSECTDIRCKHSMKYRLSFCRKTCGTC | ShK-V9/E11/D14/I15/R16 | 196 |
| RSCIDTIPVSECTDIRCKHSMAYRLSFCRKTCGTC | ShK-V9/E11/D14/ | 197 |
| | I15/R16/A22 | |
| SCIDTIPVSECTDIRCKHSMKYRLSFCRKTCGTC | ShK-V9/E11/D14/ | 198 |
| | I15/R16d1 | |
| SCIDTIPVSECTDIRCKHSMAYRLSFCRKTCGTC | ShK-V9/E11/D14/I15/ | 199 |
| | R16/A22d1 | |
| RSCIDTIPVSECTDIQCKHSMKYRLSFCRKTCGTC | ShK-V9/E11/D14/I15 | 200 |
| RSCIDTIPVSECTDIQCKHSMAYRLSFCRKTCGTC | ShK-V9/E11/D14/I15/A22 | 201 |
| SCIDTIPVSECTDIQCKHSMKYRLSFCRKTCGTC | ShK-V9/E11/D14/I15 d1 | 202 |
| SCIDTIPVSECTDIQCKHSMAYRLSFCRKTCGTC | ShK-V9/E11/D14/ | 203 |
| | I15/A22d1 | |
| RTCKDLIPVSECTDIRCKHSMKYRLSFCRKTCGTC | ShK-T2/K4/L6/V9/E11/ | 204 |
| | D14/115/R16 | |
| RTCKDLIPVSECTDIRCKHSMAYRLSFCRKTCGTC | ShK-T2/K4/L6/V9/E11/ | 205 |
| | D14/I15/R16/A22 | |
| TCKDLIPVSECTDIRCKHSMKYRLSFCRKTCGTC | ShK-T2/K4/L6/V9/E11/ | 206 |
| | D14/I15/R16 d1 | |
| TCKDLIPVSECTDIRCKHSMAYRLSFCRKTCGTC | ShK-T2/K4/L6/V9/E11/ | 207 |
| | D14/115/R16/A22 d1 | |
| (L-PhosphoTyr)-AEEAc- | ShK(L5) | 208 |
| RSCIDTIPKSRCTAFQCKHSMKYRLSFCRKTCGTC | | |
| (L-Tyr)-AEEAc- | ShK(L4) | 209 |
| RSCIDTIPKSRCTAFQCKHSMKYRLSFCRKTCGTC | | |
| (L-Tyr)-AEEAc- | ShK-198 | 210 |
| RSCIDTIPKSRCTAFQCKHSMKYRLSFCRKTCGTC- | | |
| amide | | |
| QSCADTIPKSRCTAAQCKHSMKYRLSFCRKTCGTC | ShK-Q1/A4/A15 | 211 |
| QSCADTIPKSRCTAAQCKHSMAYRLSFCRKTCGTC | ShK-Q1/A4/A15/A22 | 212 |
| QSCADTIPKSRCTAAQCKHSM(Dap)YRLSFCRKTCGTC | ShK-Q1/A4/A15/Dap22 | 213 |
| QSCADTIPKSRCTAAQCKHSMKYRASFCRKTCGTC | ShK-Q1/A4/A15/A25 | 214 |
| QSCADTIPKSRCTAAQCKHSMAYRASFCRKTCGTC | ShK-Q1/A4/A15/A22/A25 | 215 |
| QSCADTIPKSRCTAAQCKHSM(Dap)YRASFCRKTCGTC | ShK-Q1/A4/A15/ | 216 |
| | Dap22/A25 | |
| (L-PhosphoTyr)-AEEAc- | ShK-186 | 217 |
| RSCIDTIPKSRCTAFQCKHSMKYRLSFCRKTCGTC- | | |
| amide | | |
| (Para-phosphono-Phe)-AEEAc- | ShK-192 | 218 |
| RSCIDTIPKSRCTAFQCKHS(NIe)KYRLSFCRKTCGTC- | | |
| amide | | |
| (Phosphonomethyl-Phe)-AEEAc- | ShK-191 | 219 |
| RSCIDTIPKSRCTAFQCKHSMKYRLSFCRKTCGTC- | | |
| amide | | |
| (Phosphonomethyl-Phe)-AEEAc- | ShK-191/N1e21 | 220 |
| RSCIDTIPKSRCTAFQCKHS(NIe)KYRLSFCRKTCGTC- | | |
| amide | | |
| DOTA-aminohexanoicacid-(L-Tyr)-AEEAc- | ShK-221 | 221 |
| RSCIDTIPKSRCTAFQCKHSMKYRLSFCRKTCGTC- | | |
| amide | | |
| (Para-phosphono-Phe)-AEEAc- | ShK-223 | 222 |
| RSCIDTIPKSRCTAFKCKHS(NIe)KYRLSFCRKTCGTC- | | |
| amide | | |
| (Para-phosphono-Phe)-AEEAc- | ShK-190 | 223 |
| RSCIDTIPKSRCTAFQCKHSMKYRLSFCRKTCGTC | | |
| -amide | | |
| RSCIDTIPKSRCTAFQCKHS(Nle)(Dap)YRLSFCRKTCG | | 224 |
| TC | | |

Notes:
X(s1), X(s2), X(s3), etc. each refer independently to nonfunctional amino acid residues.
N-acetylR refers to N-acetylarginine
Nle refers to Norleucine
Orn refers to Ornithine
Homocit refers to Homocitrulline
NitroF refers to Nitrophenylalanine
AminoF refers to Aminophenylalanine
BenzylF refers to Benzylphenylalanine
AEEAc refers to Aminoethyloxyethyloxyacetic acid
Dap refers to Diaminopropionic acid
DOTA refers to 1,4,7,10-tetraazacyclododecane-1,4,7,10-tetraacetic acid

Toxin-based therapeutic peptides for use with the methods disclosed herein also include peptides that share: 80% identity with any of SEQ ID NO:1-251; 81% identity with any of SEQ ID NO:1-251; 82% identity with any of SEQ ID NO:1-251; 83% identity with any of SEQ ID NO:1-251; 84% identity with any of SEQ ID NO:1-251; 85% identity with any of SEQ ID NO:1-251; 86% identity with any of SEQ ID NO:1-251; 87% identity with any of SEQ ID NO:1-251; 88% identity with any of SEQ ID NO:1-251; 89% identity with any of SEQ ID NO:1-251; 90% identity with any of SEQ ID NO:1-251; 91% identity with any of SEQ ID NO:1-251; 92% identity with any of SEQ ID NO:1-251; 93% identity with any of SEQ ID NO:1-251; 94% identity with any of SEQ ID NO:1-251; 95% identity with any of SEQ ID NO:1-251; 96% identity with any of SEQ ID NO:1-251; 97% identity with any of SEQ ID NO:1-251; 98% identity with any of SEQ ID NO:1-251; or 99% identity with any of SEQ ID NO:1-251.

ShK-based peptides for use with the methods disclosed herein also include peptides that share: 80% identity with any of SEQ ID NO:1-224; 81% identity with any of SEQ ID NO:1-224; 82% identity with any of SEQ ID NO:1-224; 83% identity with any of SEQ ID NO:1-224; 84% identity with any of SEQ ID NO:1-224; 85% identity with any of SEQ ID NO:1-224; 86% identity with any of SEQ ID NO:1-224; 87% identity with any of SEQ ID NO:1-224; 88% identity with any of SEQ ID NO:1-224; 89% identity with any of SEQ ID NO:1-224; 90% identity with any of SEQ ID NO:1-224; 91% identity with any of SEQ ID NO:1-224; 92% identity with any of SEQ ID NO:1-224; 93% identity with any of SEQ ID NO:1-224; 94% identity with any of SEQ ID NO:1-224; 95% identity with any of SEQ ID NO:1-224; 96% identity with any of SEQ ID NO:1-224; 97% identity with any of SEQ ID NO:1-224; 98% identity with any of SEQ ID NO:1-224; or 99% identity with any of SEQ ID NO:1-224.

As is known in the art, “% identity” refers to a relationship between two or more peptide sequences, as determined by comparing the sequences. In the art, “identity” also means the degree of sequence relatedness between peptides as determined by the match between strings of such sequences. “Identity” (often referred to as “similarity”) can be readily calculated by known methods, including, but not limited to, those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp (1989) CABIOS. 5:151-153) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410, 1990,); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this disclosure it will be understood that where sequence analysis software is used for analysis, the results of the analysis are based on the “default values” of the program referenced. As used herein “default values” will mean any set of values or parameters which originally load with the software when first initialized.

In any of SEQ ID NO:1-251, having a Met at position 21, this Met can be replaced with Nle. In any of SEQ ID NO:1-251, having a Lys at position 22, this Lys can be replaced with diaminopropionic acid.

Embodiments disclosed herein include derivatives and analogs of the therapeutic peptides described herein. In some embodiments, derivatives and analogs have 1, 2, 3, 4, 5, 6, 7, 8, 9, 10, 11, 12, 13, 14, 15, 16, 17, 18, 19, or 20 sequence substitutions, additions, deletions, modifications, stop positions, replacements or conjugations. In additional embodiments an Xaa position can be included in any position of a peptide, wherein Xaa represents a substitution, addition, deletion, modification, stop position, replacement or conjugation position.

Each therapeutic peptide sequence disclosed herein may also include substitutions, additions, deletions, modifications, stop positions, replacements or conjugations at any position including position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of the sequence. Accordingly, in particular embodiments each amino acid position of each sequence can be an Xaa position wherein Xaa denotes a substitution, addition, deletion, modification, stop position, replacement or conjugation of the amino acid at the particular position. In particular embodiments, each sequence has 1, 2, 3, 4, 5, 6, 7, 8, 9, 10, 11, 12, 13, 14, 15, 16, 17, 18, 19, or 20 Xaa positions at one or more of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60.

The term “nonfunctional residue” refers to amino acid residues in D- or L-form having sidechains that lack acidic, basic, or aromatic groups. Exemplary nonfunctional amino acid residues include M, G, A, V, I, L and norleucine (Nle).

In particular embodiments, the C-terminus is an acid (for example, COOH) or an amide (for example, CONH₂), and the therapeutic peptide is attached to an organic or inorganic chemical entity that has an anionic charge. By “amide” it is meant the substitution of the C-terminal hydroxyl group (OH) of an acid with NH₂. Such substitution is designated herein using the term “amide” or as the C-terminal amino acid-NH2, as in “-Cys-NH₂.”

A variety of substitutions and modifications of the peptides of SEQ ID NO:1, or any of SEQ ID NO:2-251 are suitable. Therapeutic peptides can have any combination of the substitutions and modifications disclosed herein. To improve the pharmacokinetic and pharmacodynamic (PK/PD) properties of the structure of therapeutic peptides, residues that are sensitive to degradation properties can be substituted, replaced or modified. For example, a Met residue at position 21 can be substituted to impart a stabilizing effect against oxidation. In one embodiment, a Met at position 21 is substituted with Nle. Modification of the C-terminal acid function with an amide can also impart stability. These changes to the primary structure of therapeutic peptides can be combined with an anionic moiety at the N-terminus to produce a stable and selective Kv1.3 blocker. Accordingly, one embodiment disclosed herein includes SEQ ID NO:1 wherein the methionine at position 21 is substituted with Nle, an amide is present at the C-terminus and/or an anionic moiety is present at the N-terminus.

The safety, potency, and specificity of a variety of therapeutic peptides have been investigated and attaching the peptide to an organic or inorganic chemical entity that has an anionic charge has been shown to improve the suitability for use in pharmaceutical compositions.

Those skilled in the art are aware of techniques for designing therapeutic peptides with enhanced properties, such as alanine scanning, rational design based on alignment mediated mutagenesis using known sequences and/or molecular modeling. For example, therapeutic peptides can be designed to remove protease cleavage sites (e.g., trypsin cleavage sites at K or R residues and/or chymotrypsin cleavage sites at F, Y, or W residues) in a therapeutic peptide-containing composition. Nonhydrolyzable phosphate substitutions also impart a stabilizing effect on the phosphate groups, as well as stability against phosphatase enzymes.

Certain embodiments include the attachment of an organic or inorganic chemical entity. The site of attachment can be the N-terminus, but modifications are not limited to attachment at this site. Exemplary chemical entities can be attached by way of a linker, such as an aminoethyloxyethyloxy-acetyl linker (referred to herein as AEEAc), or by any other suitable means.

Examples of appropriate chemical entities include L-Pmp(OH₂); D-Pmp(OH₂); D-Pmp(OHEt); Pmp(Et₂); D-Pmp(Et₂); L-Tyr; L-Tyr(PO₃H₂) (p-phospho-Tyrosine); L-Phe(p-NH₂); L-Phe(p-CO₂H); L-Aspartate; D-Aspartate; L-Glutamate; and D-Glutamate. The abbreviations used are defined as follows: Pmp (p-phosphonomethyl-phenylalanine); and Ppa (p-phosphatityl-phenylalanine). Alternatives to PmP and Ppa include, without limitation, Pfp (p-Phosphono(difluoro-methyl)-Phenylalanine) and Pkp (p-Phosphono-methylketo-Phenylalanine).

Examples of chemical entity/linker combinations include AEEAc-L-Pmp(OH₂); AEEAc-D-Pmp(OH₂); AEEAc-D-Pmp(OHEt); AEEAc-L-Pmp(Et₂); AEEAc-D-Pmp(Et₂); AEEAc-L-Tyr; AEEAc-L-Tyr(PO₃H₂); AEEAc-L-Phe(p-NH₂); AEEAc-L-Phe(p-CO₂H); AEEAc-L-Aspartate; AEEAc-D-Aspartate; AEEAc-L-Glutamate; and AEEAc-D-Glutamate. In the chemical entities generally, where the amino acid residue has a chiral center, the D and/or L enantiomer of the amino acid residue can be used.

For a therapeutic peptide to find use as a pharmaceutical composition, it must meet several criteria in terms of stability, solubility, and pH, and preferably only contain materials consistent with administration to animals including, without limitation, mammals, and particularly humans. The composition can be varied depending on the mode of administration, such as subcutaneous, intravenous, etc.

As used herein, a “pharmaceutical composition” comprises at least one therapeutic peptide together with one or more pharmaceutically acceptable carriers, excipients, or diluents, as appropriate for the chosen mode of administration.

Embodiments disclosed herein can be used to effectively treat IPEX and its associated autoimmune disorders associated with Kv1.3 channel binding.

ShK-186 is a potent and specific inhibitor of the Kv1.3 potassium channel. The family of ShK-based molecules, including ShK-186, provides a new treatment option for IPEX subjects that could reduce autoimmune activation with a more favorable safety profile than existing therapies. One particular formulation of ShK-186 with optimal stability and solubility for use in the methods disclosed herein (referred to as P6N) comprises, consists of or consists essentially of the components shown in Table 3:

TABLE 3
Pharmaceutical composition of P6N formulation

| Component | Concentration | Purpose |
|---|---|---|
| ShK-186 peptide | Up to 500 mg/mL | Active agent |
| Sodium phosphate | 10 mM | Buffering agent |
| NaCl | 150 mM | Tonicity modifier |
| Polysorbate 20 | 0.05% (w/v) | Surfactant |
| pH of 6.0 | | |

Other therapeutic peptides can be used with the same, or similar, formulations.

Methods disclosed herein include treating subjects (humans, veterinary animals, livestock, and research animals) with pharmaceutical compositions. Treating subjects includes delivering an effective amount or delivering a prophylactic treatment and/or a therapeutic treatment. An “effective amount” is the amount of a compound necessary to result in a desired physiological change in the subject such as a prophylactic treatment or a therapeutic treatment. Effective amounts are often also administered for research purposes. Effective amounts may reduce the population of T_{EM} (i.e., reduce proliferation); reduce activation of T_{EM} as measured by cytokine production (e.g., IFN-γ; IL-2; IL-4; IL-10; IL-17 and IL-21) and/or perforin production; and/or reduce expression of Kv1.3 channels. Reductions can be seen based on comparisons to a previous measure from the same subject or as compared to comparisons to a reference population not affected by IPEX.

Effective amounts may reduce the occurrence and/or symptoms of conditions and diseases related to, or caused by, IPEX. In an IPEX subject presenting with diabetes, an effective amount may be an amount that helps to maintain the subject's blood glucose level in their target range, for example 70 to 130 mg/dL before meals and less than 180 mg/dL 1 to 2 hours after the start of a meal.

In subjects with IPEX-related eczema, an effective amount may be an amount that results in a 50% reduction in the Eczema Assessment Severity Index (EASI), a 55% reduction in the EASI, a 60% reduction in the EASI, a 65% reduction in the EASI, a 70% reduction in the EASI, a 75% reduction in the EASI, a 80% reduction in the EASI, a 85% reduction in the EASI, a 90% reduction in the EASI, a 95% reduction in the EASI, or a 100% reduction in the EASI.

In subjects with IPEX-related thyroid dysfunction (hypo or hyper-thyroidism), an effective amount may be an amount that results in the subject's thyroid-stimulating hormone (TSH) levels being closer to a normal range as compared to previous measurements from the same subject or as compared to a reference population that is not affected by IPEX.

As these methods of evaluating effective amounts indicate, those of ordinary skill in the art are familiar with numerous clinical assessments for secondary conditions caused by or related to underlying IPEX including diabetes, hypo- or hyperthyroidism, enteropathy, inflammation, dermatitis, eczema, polyendocrinopathy, anemia, thrombocytopenia, neutropenia, hepatitis, tubular nephropathy, spenomegaly, alopecia and infections.

A “prophylactic treatment” includes a treatment administered to a subject who does not display signs or symptoms of IPEX or a disease or condition associated with or caused by IPEX, or displays only early signs or symptoms of the disease or condition, such that treatment is administered for the purpose of diminishing, preventing, or decreasing the risk of developing the disease or condition further. Thus, a prophylactic treatment functions as a preventative treatment against IPEX or a disease or disorder associated with or caused by IPEX.

A “therapeutic treatment” includes a treatment administered to a subject who displays symptoms or signs of IPEX or a disease or condition associated with, or caused by, IPEX, and is administered to the subject for the purpose of diminishing or eliminating those signs or symptoms of IPEX or the related disease or condition.

“Therapeutically effective amounts” include those that provide prophylactic treatment and/or therapeutic treatment. Therapeutically effective amounts need not fully prevent or cure IPEX or the related disease or condition, but can also provide a partial benefit, such as alleviation or improvement of at least one symptom of IPEX or an IPEX-related disease or condition.

Treating IPEX includes administering an effective amount and/or a therapeutically effective amount.

For administration, effective amounts and therapeutically effective amounts (also referred to herein as doses) can be initially estimated based on results from in vitro assays and/or animal model studies. For example, a dose can be formulated in animal models to achieve a circulating concentration range that includes an IC₅₀ as determined in cell culture against activation, proliferation, cytokine production and/or perforin production by T_{EM}. Such information can be used to more accurately determine useful doses in subjects of interest.

The actual dose amount administered to a particular subject can be determined by a physician, veterinarian, or researcher taking into account parameters such as physical and physiological factors including body weight, severity of condition, age, diet of the subject, sex, severity of the condition, time of administration, type of disease, previous or concurrent therapeutic interventions, idiopathy of the subject, the route of administration, and other clinically relevant factors.

Useful doses often range of 1 to 10,000 micrograms (μg) of the therapeutic peptide per kilogram (kg) of body mass, in the range of 1 to 5,000 μg per kilogram of body mass, in the range of 1 to 1,000 μg per kg of body mass or in the range of 1 to 100 μg per kg of body mass. Often, doses may range from 0.1 to 5 μg/kg or from 0.5 to 1 μg/kg. In other examples, a dose can comprise 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1,000 μg/kg, 1,500 μg/kg, 2,000 μg/kg, 2,500 μg/kg, 3,000 μg/kg, 3,500 μg/kg, 4,000 μg/kg, 5,000 μg/kg, 6,000 μg/kg, 7,000 μg/kg, 8,000 μg/kg, 9,000 μg/kg, 10,000 μg/kg, 0.1 to 5 mg/kg or from 0.5 to 1 mg/kg. In other examples, a dose can comprise 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, or 50 mg/kg.

Effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly.)

For administration of the pharmaceutical composition, a suitable route is injection. A medical practitioner will be familiar with methods of administration through injection depending on the subject and the type of injection, such as subcutaneous, intravenous, etc. U.S. Pat. No. 7,918,824 discloses syringes suitable for subject use. The therapeutic peptides can be formulated for parenteral administration by injection e.g. by bolus injection or infusion.

Formulations for injection can be presented in unit dosage form, e.g. in glass ampoule or multi dose containers, e.g. glass vials. The compositions for injection can take such forms as suspensions, solutions or emulsions in oily or aqueous vehicles, and can contain formulatory agents such as suspending, stabilising, preserving, and/or dispersing agents. Alternatively, the active ingredient can be in powder form for constitution with a suitable vehicle, e.g. sterile pyrogen-free water, before use.

Oral administration of the therapeutic peptides requires formulations that do not allow degradation of the peptides by digestive mechanisms before producing a desired physiological change. Solid dosage forms for oral administration can include capsules, tablets, pills, powders, and granules. In such solid dosage forms, the therapeutic peptides can be admixed with at least one inert diluent such as sucrose, lactose, or starch. Such dosage forms can also comprise, as in normal practice, additional substances other than inert diluents, e.g., lubricating agents such as magnesium stearate. In the case of capsules, tablets, and pills, the dosage forms can also comprise buffering agents. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting, sweetening, flavoring, and perfuming agents. The pharmaceutical composition can contain more than one embodiment of the present disclosure. Preparations for oral administration can be suitably formulated to give controlled release of the therapeutic peptides.

For buccal administration the compositions can take the form of tablets or lozenges formulated in conventional manners.

In addition to the formulations described above, therapeutic peptides can also be formulated as depot preparations. Such long acting formulations can be administered by implantation or by intramuscular injection.

For nasal or pulmonary administration or any other administration by inhalation, the therapeutic peptides can be conveniently delivered in the form of an aerosol spray presentation for pressurized packs or a nebulizer, with the use of suitable propellant, e.g. dichlorodifluoromethane, trichlorofluoromethane, dichlorotetrafluoroethane, carbon dioxide or other suitable gas or mixture of gases.

The Examples below were designed to characterize T cell populations expanded in IPEX subjects immunophenotypically and to determine their expression of and functional dependence on the Kv1.3 channel. The results can identify individual subjects and subject populations amenable for treatment with therapeutic peptides as described herein.

The Examples below describe the optimization of the methods disclosed herein. These Examples are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXAMPLES

### Example 1

### Expression of Kv1.3 Channels by Effector Memory T Cells

This example is performed to evaluate the expression of Kv1.3 channels by T_{EM} found in IPEX subjects compared to controls, and to characterize the responsiveness of IPEX subject samples to treatment with therapeutic peptides such as the ShK-based peptide, ShK-186, formulated into a pharmaceutical composition. To evaluate Kv1.3 expression, peripheral blood mononuclear cells from IPEX and healthy controls are isolated. In the first phase of the example, the cells are activated in vitro with anti-CD3 antibodies or mitogens and then subjected to immunostaining and multicolor flow cytometry.

In the second phase, matching cell aliquots are analyzed by RT-QPCR for quantitation of Kv1.3 mRNA. In the third phase, matching cell aliquots are used to study the ability of ShK-186 to block the proinflammatory and proliferative potential of T_{EM} from IPEX subjects versus controls. Cytokines (IFN-γ and IL-2, -4, -10, -17, and -21) and perforin are detected by intracellular cytokine staining and flow cytometry in resting or activated cells in the presence or absence of ShK-186. In this example, ten IPEX and ten normal controls are studied.

### Example 2

### Fluorescence Activated Cell Sorter (FACS) Analysis

Peripheral blood mononuclear cells (PBMCs) are evaluated by multiparameter flow cytometry for cell surface expression of CD4 and CD25 and intracellular FOXP3. FACS sorting of all stimulated and stained samples will also be performed.

The results will demonstrate that ShK-186 reduces (i) expression of Kv1.3 channels by T_{EM}; (ii) IFN-γ and IL-2, -4, -10, -17, and/or -21 production by T_{EM}; and/or (iii) proliferation of T_{EM}.

Additional studies will demonstrate that administering effective amounts of the therapeutic peptides disclosed herein, including in particular embodiments, Shk-186, produces clinically relevant improvements in IPEX and secondary conditions related to IPEX such as one or more of diabetes, hypo- or hyperthyroidism, enteropathy, inflammation, dermatitis, eczema, polyendocrinopathy, anemia, thrombocytopenia, neutropenia, hepatitis, tubular nephropathy, spenomegaly, alopecia and infections

### Exemplary Embodiments

1. A method of treating IPEX in a subject in need thereof comprising administering to the subject a pharmaceutical composition selected from:
(a) an ShK peptide having the sequence SEQ ID NO:1 and/or SEQ ID NO:49, wherein the ShK peptide is attached to an organic or inorganic chemical entity that has an anionic charge, and/or the C-terminus is an acid or an amide;
(b) an ShK peptide having the formula SEQ ID NO:217;
(c) an ShK peptide having the formula SEQ ID NO:210; or
(d) a combination of (a), (b), and/or (c) in an effective amount thereby treating the subject.
2. The method of embodiment 1, wherein the pharmaceutical composition comprises a pharmaceutically acceptable salt of the administered ShK peptide(s).
3. The method of embodiments 1 or 2, wherein the pharmaceutical composition is administered daily, weekly, monthly, every two months, every three months, or every six months.
4. The method of embodiments 1, 2 or 3, wherein the pharmaceutical composition is administered subcutaneously or intravenously.
5. A method of treating IPEX in a subject in need thereof comprising administering to the subject a pharmaceutical composition comprising 10 mM sodium phosphate; 150 mM NaCl; 0.5 mg/ml to 50 mg/ml of a pharmaceutically acceptable acetate salt of a peptide consisting of SEQ ID NO:217; and Polysorbate 20 at 0.05 w/v %, wherein the composition has a pH of 6.0 in an effective amount thereby treating the subject.
6. The method of embodiment 5, wherein the pharmaceutical composition is administered daily, weekly, monthly, every two months, every three months, or every six months.
7. The method of embodiments 5 or 6, wherein the pharmaceutical composition is administered subcutaneously or intravenously.
8. A method of evaluating a subject to assess the probable outcome of treatment with a method of any of embodiments 1, 2, 3, 4, 5, 6 or 7 comprising analyzing a biological sample from the subject for T cell populations and levels of expression of Kv1.3 on said T cell populations wherein the ability of the pharmaceutical composition to block the proinflammatory and proliferative potential of T cells predicts a probable positive outcome to the treatment.
9. The method of embodiment 8, wherein said T cells are Memory T cells.
10. The method of embodiments 8 or 9, wherein said biological sample consists of peripheral blood mononuclear cells.
11. The method of embodiments 8, 9 or 10, wherein said proinflammatory and proliferative potential is determined by measuring the secretion of at least one cytokine from the Memory T cells.
12. The method of embodiment 11, wherein said cytokine is selected from IFN-γ, IL-2, IL-4, IL-10, IL-17, or IL-21.
13. The method of embodiments 8, 9, 10, 11 or 12 wherein the T cells are T_{EM}.
14. A method of treating IPEX in a subject in need thereof comprising administering to the subject a pharmaceutical composition comprising a toxin-based therapeutic peptide in an effective amount, thereby treating the subject.
15. The method of embodiment 14, wherein the toxin-based therapeutic peptide has a sequence selected from SEQ ID NO:225-251.
16. The method of embodiments 14 or 15, wherein the pharmaceutical composition comprises a pharmaceutically acceptable salt of the toxin-based therapeutic peptide.
17. The method of embodiments 14 15 or 16, wherein the pharmaceutical composition is administered daily, weekly, monthly, every two months, every three months, or every six months.
18. The method of embodiments 14, 15, 16, or 17, wherein the pharmaceutical composition is administered subcutaneously or intravenously.
19. A method of treating IPEX in a subject in need thereof comprising administering to the subject a pharmaceutical composition comprising 10 mM sodium phosphate; 150 mM NaCl; 0.5 mg/ml to 50 mg/ml of a pharmaceutically acceptable acetate salt of a toxin-based therapeutic peptide; and Polysorbate 20 at 0.05 w/v %, wherein the composition has a pH of 6.0, in an effective amount thereby treating the subject.
20. The method of embodiment 17, wherein the pharmaceutical composition is administered daily, weekly, monthly, every two months, every three months, or every six months.
21. The method of embodiments 17 or 18, wherein the pharmaceutical composition is administered subcutaneously or intravenously.
22. A method of treating IPEX in a subject in need thereof comprising administering to the subject a pharmaceutical composition comprising an ShK-based peptide in an effective amount, thereby treating the subject.
23. The method of embodiment 22, wherein the ShK-based peptide has a sequence selected from SEQ ID NO: 1-224.
24. The method of embodiments 22 or 23, wherein the pharmaceutical composition comprises a pharmaceutically acceptable salt of the ShK-based peptide.
25. The method of embodiments 22, 23 or 24, wherein the pharmaceutical composition is administered daily, weekly, monthly, every two months, every three months, or every six months.
26. The method of embodiments 22, 23, 24, or 25, wherein the pharmaceutical composition is administered subcutaneously or intravenously.
27. A method of treating IPEX in a subject in need thereof comprising administering to the subject a pharmaceutical composition comprising 10 mM sodium phosphate; 150 mM NaCl; 0.5 mg/ml to 50 mg/ml of a pharmaceutically acceptable acetate salt of a ShK-based peptide; and Polysorbate 20 at 0.05 w/v %, wherein the composition has a pH of 6.0, in an effective amount thereby treating the subject.
28. The method of embodiment 27, wherein the pharmaceutical composition is administered daily, weekly, monthly, every two months, every three months, or every six months.
29. The method of embodiments 27 or 28, wherein the pharmaceutical composition is administered subcutaneously or intravenously.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically significant reduction in the effectiveness of an IPEX-related treatment selected from TEM expression of Kv1.3 channels; TEM proliferation, TEM production of cytokines selected from one or more of IFN-γ, IL-2, IL-4, IL-10, IL-17, or IL-21, production of perforin and/or a clinically relevant treatment parameter associated with diabetes, hypo- or hyperthyroidism, enteropathy, inflammation, dermatitis, eczema, polyendocrinopathy, anemia, thrombocytopenia, neutropenia, hepatitis, tubular nephropathy, spenomegaly, alopecia and infections

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1.-29. (canceled)

30. A method of treating immune dysregulation, polyendocrinopathy, enteropathy, X-linked syndrome (IPEX) in a subject in need thereof, the method comprising:
administering to the subject an effective amount of a pharmaceutical composition comprising one or more peptides having sequences selected from SEQ ID NO: 1-251, thereby treating IPEX in the subject.

31. The method of claim 30, wherein the one or more peptides are ShK-based peptides having sequences selected from SEQ ID NO:1-224.

32. The method of claim 31, wherein the sequences are selected from:
(a) SEQ ID NO:1, wherein the ShK-based peptide is attached to an organic or inorganic chemical entity that has an anionic charge, and wherein the C-terminus is an acid or an amide;
(b) SEQ ID NO:49, wherein the ShK-based peptide is attached to an organic or inorganic chemical entity that has an anionic charge, and wherein the C-terminus is an acid or an amide;
(c) SEQ ID NO:217;
(d) SEQ ID NO:210;
(e) SEQ ID NO:218; or
(f) a combination of (a), (b), (c), (d), and/or (e).

33. The method of claim 32, wherein the organic or inorganic chemical entity is selected from the group consisting of: L-Pmp(OH₂); D-Pmp(OH₂); D-Pmp(OHEt); Pmp(Et₂); D-Pmp(Et₂); L-Tyr; L-Tyr(PO₃H₂); L-Phe(p-NH₂); L-Phe(p-CO₂H); L-Aspartate; D-Aspartate; L-Glutamate; D-Glutamate; Ppa; Pfp; and Pkp.

34. The method of claim 33, wherein the ShK-based peptide is attached to the organic or inorganic chemical entity by an aminoethyloxyethyloxy-acetyl linker (AEEAc).

35. The method of claim 30, wherein the sequences are selected from SEQ ID NO:225-251.

36. The method of claim 30, wherein the pharmaceutical composition comprises a pharmaceutically acceptable salt of the one or more peptides.

37. The method of claim 36, wherein the pharmaceutical composition comprises 10 mM sodium phosphate; 150 mM NaCl; 0.5 mg/ml to 50 mg/ml of a pharmaceutically acceptable acetate salt of the one or more peptides; and 0.05 w/v % Polysorbate 20, wherein the composition has a pH of about 6.0.

38. The method of claim 32, wherein the ShK-based peptide has the sequence of SEQ ID NO:217.

39. A method of predicting an outcome of a treatment of IPEX in a subject afflicted with IPEX, the method comprising:
analyzing a biological sample from the subject for T cell populations and levels of expression of Kv1.3 channels on the T cell populations,
wherein the treatment comprises administering to the subject an effective amount of a pharmaceutical composition comprising one or more ShK-based peptides having sequences selected from SEQ ID NO: 1-224, and
wherein an ability of the pharmaceutical composition to block proinflammatory and proliferative potential of T cells predicts a positive outcome of the treatment.

40. The method of claim 39, wherein the T cell populations are memory T cell populations.

41. The method of claim 39, wherein the proinflammatory and proliferative potential is determined by measuring secretion levels of at least one cytokine from Memory T cells.

42. A method of evaluating efficacy of a treatment of IPEX in a subject, the method comprising:
analyzing a biological sample from the subject for T cell populations and levels of expression of Kv1.3 channels on the T cell populations after the subject has received the treatment,
comparing the levels of expression of Kv1.3 channels on the T cell populations to a reference level;
wherein a decrease in the levels of expression of Kv1.3 channels by T cells in the subject compared to the reference level indicates that the treatment is effective, and
wherein the treatment comprises administering to the subject an effective amount of a pharmaceutical composition comprising one or more ShK-based peptides having sequences selected from SEQ ID NO: 1-224.

43. The method of claim 42, wherein the T cell populations are Memory T cell populations.

44. The method of claim 42, wherein the reference level is (i) a measurement from the subject prior to the subject receiving the treatment or (ii) a reference score derived from a population of individuals not affected by IPEX.

45. The method of claim 42, further comprising analyzing the biological sample from the subject for activation, proliferation, cytokine production, or perforin production by Memory T cells, wherein a decrease in activation, proliferation, cytokine production, or perforin production by Memory T cells indicates that the treatment is effective.

46. The method of claim 45, wherein the analysis of cytokine production is a measurement of one or more cytokines selected from IFN-γ, IL-2, IL-4, IL-10, IL-17, and IL-21.

47. The method of claim 42, wherein the one or more ShK-based peptides has a sequence of SEQ ID NO:217.

48. The method of claim 42, further comprising evaluating one or more clinically known parameters related to one or more conditions associated with IPEX.
